(19) United States
(12) Patent Application Publication
(10) Pub. No.: US 2026/0355180 A1
(43) Pub. Date: Nov. 12, 2026

(54) COMPOSITIONS AND METHODS FOR SPLICEOSOME-TARGETED THERAPIES

(71) Applicant: Institute for Cancer Research d/b/a The Research Institute of Fox Chase Cancer Center, Philadelphia, PA (US)

(72) Inventors: Israel CAÑADAS, Meadowbrook, PA (US); Xinpei JIANG, Philadelphia, PA (US)

(73) Assignee: Institute for Cancer Research d/b/a The Research Institute of Fox Chase Cancer Center, Philadelphia, PA (US)

(21) Appl. No.: 19/134,961

(22) PCT Filed: Dec. 1, 2023
(86) PCT No.: PCT/US2023/082139

§ 371 Date: Jun. 2, 2025

Related U.S. Application Data
(60) Provisional application No. 63/385,733, filed on Dec. 1, 2022.
(60) Provisional application No. 63/599,911, filed on Nov. 16, 2023.

(51) Int. Cl. A61K 31/496; A61K 31/35; A61K 31/365; A61K 31/404; A61K 39/00; A61K 45/06; A61P 35/00; C07K 16/28

(52) U.S. Cl. CPC A61K 31/496; A61K 31/35; A61K 31/365; A61K 31/404; A61K 45/06; A61P 35/00; C07K 16/2818; A61K 2039/505

(57) ABSTRACT

Compositions and methods for the treatment of cancers using spliccosome inhibitors are provided herein.

### CROSS REFERENCE TO RELATED APPLICATIONS

This patent application claims the benefit of U.S. Provisional Patent Application No. 63/385,733, filed Dec. 1, 2022, and U.S. Provisional Patent Application No. 63/599,911, filed Nov. 16, 2023. The entire contents of each of the foregoing applications are incorporated herein by reference, including all text, tables, drawings, and sequences.

### FIELD OF THE INVENTION

The present invention relates to the field of cancer treatments. More specifically, the invention provides methods and compositions for inducing necroptosis and apoptosis in cancer cells using spliceosome-targeted therapies.

### BACKGROUND OF THE INVENTION

Several publications and patent documents are cited throughout the specification in order to describe the state of the art to which this invention pertains. Each of these citations is incorporated by reference herein as though set forth in full.

Lung cancer is the leading cause of cancer mortality in the United States with approximately 350 deaths per day¹. Small cell lung cancer (SCLC) is an aggressive high-grade neuroendocrine tumor that accounts for 15% of all lung cancers and is associated with cigarette smoking, poor prognosis, rapid metastases, and resistance to therapies. SCLCs are immune “COLD” cancers categorized by low immune infiltrates and evasion from the host immune responses.^{20,57} Patients with SCLC have 5-year overall survival of approximately 5%²⁻⁸. Despite extensive research efforts in understanding the progression and molecular feature of the disease, no effective therapies are available. Furthermore, immunotherapy has shown a durable response in tumor⁹, but only a small fraction of SCLC patients respond. Additionally, the patient median overall survival is minimal at best, only 2-3 months.¹⁰⁻¹⁵ Thus, chemotherapy remains the standard-of-care¹⁶. To date, SCLC remains a recalcitrant malignancy⁴ and devastating disease, with a continual lack of efficacious therapy options. Clearly there is an unmet need to urgently identify novel and effective targeted therapies for metastatic SCLC.

Cancer can acquire mutations that drive growth, fitness, disease progression, and resistance. SCLC is a heterogenous and highly proliferative malignancy with a high prevalence of somatic mutations^{17,18}, resistance to apoptosis¹⁹, and high tumor mutational burden (TMB)²⁰. High TMB cancers are associated with high neoantigen frequency, however, SCLCs exhibit low to zero expression of neoantigens²¹⁻²³. Lack of antigen presentation results in poor immune checkpoint blockade (ICB) therapies and through immune evasion,²⁴ while elevated antigen expression directly correlates with higher efficacy of ICB treatment²⁵. To date no durable targeted approach is available, thus, it is crucial to uncover new pathways and vulnerabilities to induce tumor-intrinsic immunogenicity to synergize with ICB for effective and durable anti-tumor immunity in SCLC patients.

### SUMMARY OF THE INVENTION

In accordance with the present invention, methods for treating a subject having tumors comprising administering to said subject at least one spliceosome modulator are provided. In certain embodiments, administration of these modulators reduces tumor cell proliferation or induces tumor cell killing which exceeds that observed in tumor cells not treated with the modulator. In a preferred embodiment, the spliceosome modulator is selected from a SF3b inhibitor, an SF3B1 inhibitor, or a TIME modulator. In certain embodiments, the spliceosome modulator is selected from pladienolide D, pladienolide B, pladienolide A, pladienolide C, pladienolide E, pladienolide F, pladienolide G, H3B-8800, indisulam, herboxidiene, spliceostatin, sudemycin, FR901463, FR901464, FR901465, splicostatins A-G, thailanstatins A-C, meamycins, E7107, FD-895, herboxidiene, GEX1A, GEX1Q1-5, RON-18690A (18-deoxyherboxidiene), sudemycins C1, sudemycins D1, sudemycins F1, sudemycins E, sudemycins D6, isoginkgetin, madrasin, tetrocarcin A, N-palmitoyl-L-leucine, psoromic acid, clotrimazole, NSC635326, napthazarin and/or derivatives thereof.

In another embodiment, the method further comprises administering at least one additional cancer treatment or therapeutic agent. In certain embodiments, this additional therapeutic agent or therapy is immunotherapy, TNF therapy, chemotherapy, radiotherapy, and/or a PD-L1 inhibitor. In certain embodiments, the tumor is a recalcitrant tumor or a COLD cancer. The tumors treated using the methods disclosed herein may be a lung cancer, such as Small Cell Lung Cancer (SCLC). In certain embodiments, the modulators arc administered in a pharmaceutically acceptable carrier via route selected from systemic, oral, intraperitoneal, intravenous, intracerebral, intratumoral and topical administration.

Another aspect of the invention includes methods for inducing ZBP1-dependent necroptosis in tumor cells comprising contacting the cells with at least one spliceosome modulator, thereby reducing tumor cell proliferation or inducing tumor cell killing which exceeds that observed in tumor cells not treated with the modulator.

Yet another aspect of the invention includes methods of generating Z-nucleic acids (Z-NA) and A-RNA species comprising, a) contacting a tumor cell with at least one spliceosome modulator, and b) separating the Z-NA and A-RNA molecules from the cells.

Another aspect of the invention includes methods of identifying endogenous Z-NA and A-RNA biomarkers, the methods comprising a) treating tumor cells with a spliceosome modulator, b) releasing immunogenic nucleic acids produced by said cells, and c) characterizing the immunogenic nucleic acids of step b). The immunogenic nucleic acids identified by these methods are enriched when compared to an untreated control, and thereby provide new biomarkers for said tumor cells. In certain embodiments the methods further comprise compiling the biomarkers into a biomarker profile for said tumor cells. In certain embodiments nucleic acids encoding the biomarkers identified herein are used RNA based silencing methods for treating a patient with an aggressive tumor.

Also provided herein are methods enhancing tumor vulnerability to chemotherapy in a tumor with a low mutational burden, the method comprising administering at least one spliceosome modulator, thereby reducing tumor cell proliferation. In certain embodiments, the method further comprises administering at least one chemotherapy or radiotherapy, thereby inducing tumor cell killing which exceeds that observed in tumor cells treated with the spliceosome modulator alone. In certain embodiments the at least one chemotherapy, or radiotherapy and at least one spliceosome modulator act synergistically.

Also provided herein are methods of enhancing tumor vulnerability to immunotherapy in a tumor with a low mutational burden, the method comprising administering at least one spliceosome modulator, thereby reducing tumor cell proliferation. In certain embodiments, the method further comprises administering at least one immunotherapy, thereby inducing tumor cell killing which exceeds that observed in tumor cells treated with the spliceosome modulator alone. In certain embodiments the at least one immunotherapy and at least one spliccosome modulator act synergistically.

In certain embodiments of the above methods, the at least one spliceosome modulator is selected from a SF3b inhibitor, an SF3B1 inhibitor, or a TIME modulator. In certain embodiments, the spliceosome modulator is selected from at least one of pladienolide D, pladienolide B, pladienolide A, pladienolide C, pladienolide E, pladienolide F, pladienolide G, H3B-8800, indisulam, herboxidiene, spliceostatin, sudemycin, FR901463, FR901464, FR901465, splicostatins A-G, thailanstatins A-C, meamycins, E7107, FD-895, herboxidiene, GEX1A, GEX1Q1-5, RQN-18690A (18-deoxyherboxidiene), sudemycins C1, sudemycins D1, sudemycins F1, sudemycins E, sudemycins D6, isoginkgetin, madrasin, tetrocarcin A, N-palmitoyl-L-leucine, psoromic acid, clotrimazole, NSC635326, napthazarin and/or derivatives thereof. In certain embodiments, the chemotherapy or immunotherapy is a PD-L1 or PD-1 inhibitor.

In certain embodiments of the above methods, the tumor is a recalcitrant tumor or a COLD cancer. The tumors treated using the methods disclosed herein may be a lung cancer, such as Small Cell Lung Cancer (SCLC). In certain embodiments, the modulators are administered in a pharmaceutically acceptable carrier via route selected from systemic, oral, intraperitoneal, intravenous, intracerebral, intratumoral and topical administration.

Another aspect of the invention includes methods for inducing ZBP1-dependent necroptosis in tumor cells comprising contacting the cells with at least one spliceosome modulator and at least one immunotherapy, thereby reducing tumor cell proliferation or inducing tumor cell killing which exceeds that observed in tumor cells treated with the modulator alone. In certain embodiments the at least one immunotherapy and at least one spliceosome modulator act synergistically.

Yet another aspect of the invention comprises methods for inducing ZBP1-dependent necroptosis in Small Cell Lung Cancer (SCLC) tumor cells, the method comprising administering to said subject effective amounts at least one spliceosome modulator and at least one immunotherapy, thereby reducing tumor cell proliferation or inducing tumor cell killing which exceeds that observed in tumor cells treated with the modulator alone, wherein said spliceosome modulator is selected from pladienolide B, H3B-8800, sudemycin D6, and indisulam; and said immunotherapy is a PD-1 inhibitor or PD-L1 inhibitor. In certain embodiments the immunotherapy and the at least one spliccosome modulator act synergistically.

In certain embodiments, the spliceosome modulator is plabienolide B administered a) via intratumoral administration at a dose of 0-200 nM; b) via intraperitoneal administration at a dose of 0-20 mg/kg; or c) via systemic or intravenous administration at a dose of 2-20 mg/kg. In certain embodiments, the spliceosome modulator is H3B-8800 administered orally at a dose of 0-40 mg. In certain embodiments, the spliceosome modulator is sudemycin D6 administered via systemic or intravenous administration at a dose of 14 mg/kg-50 mg/kg. In certain embodiments, the spliceosome modulator is indisulam administered via systemic or intravenous administration at a dose of 12.5 mg/kg-100 mg/kg. In certain embodiments, the SCLC is a COLD cancer and said modulators are administered in a pharmaceutically acceptable carrier via route selected from systemic, oral, intraperitoneal, intravenous, intracerebral, intratumoral and topical administration, said method optionally comprising administration of a radiotherapy.

In certain embodiments of the methods above, the method further comprises administering at least one TNFR1, TRADD, FADD, or CASP8 inhibitor.

### BRIEF DESCRIPTION OF THE DRAWINGS

FIG. 1: Schematic of Pladienolide B (PlaB)-mediated antiviral response and schematic diagram showing effects of treatment protocol hypotheses. PlaB induces multiple A-RNAs and Z-NAs that in turn trigger both necroptosis and apoptosis in targeted cells.

FIG. 2A-2C: SF3B1 inhibition dramatically reduces SCLC cell survival and RIPK1 depletion rescues PlaB mediated cytotoxicity. FIG. 2A) SCLC cell lines (H446 and H82) and immortalized fibroblasts (FC1010 and HS68) cell survival curves after 72 hours of PlaB treatment. FIG. 2B) RIPK1 depletion rescues H82 and H446 cell survival after 18 hours of PlaB treatment. FIG. 2C) Western blot showing RIPK1 protein depletion. Mean±SEM, Welch's t test *p<0.05, **p<0.01.

FIG. 3A-3D: SF3B1 inhibition induces a potent IFN response and increases expression of IFN-regulated proteins. (FIG. 3A and FIG. 3B) PlaB induces potent IFN response in SCLC cell lines, H446 and H82, in a dose-dependent manner. (FIG. 3C and FIG. 3D) 48 hours of 0.5 nM of PlaB treatment effectively upregulates antigen and PD-L1 expression in H446 and H82 SCLC cell lines. Mean±SEM, two-tailed unpaired Student's t test *p<0.05, **p<0.01, ***p<0.001, ****p<0.0001.

FIG. 4A-4C: SF3B1 perturbation increases dsRNA and Z-nucleic acid levels in treated cells. (FIG. 4A) Low dose of PlaB and indisulam induces accumulation of A-RNA and Z-Nucleic acids (mouse J2 and rabbit Z22 antibodies respectively) in H446. (FIG. 4B) Quantification of A-RNA and Z-Nucleic acids. (FIG. 4C) Western blot showing induction of cleaved-PARP, pH2AX, and ZBP1, and the reduction of RIPK1 protein levels. Plots of signal intensity are mean±SEM, two-tailed unpaired Student's t test **p<0.01, ****p<0.0001.

FIG. 5A-5E: SF3B1 perturbation upregulates A-RNA and Z-nucleic acids in MEFs. (FIG. 5A) 5 nM of PlaB induces A-RNA and Z-Nucleic acids in ΔZBP1 MEF cells. (FIG. 5B) Quantification of A-RNA and Z-Nucleic acids. (FIG. 5C) RIPK3 inhibitor, GSK-843, successfully rescue PlaB mediated necroptosis in ZBP1 reconstituted MEF. (FIG. 5D) Western blot showing induction of pMLKL only in MEF expressing full-length ZBP1. (FIG. 5E) MEF ΔZα and RHIM-A mutants avoid PlaB-mediated necroptosis. Plots of signal intensity are mean±SEM, two-tailed unpaired Student's t test *p<0.05, ***p<0.001, ****p<0.0001. 18 hours of PlaB treatment for IF, WB and rescue experiments. IF: 5 nM PlaB, WB and rescue experiments: 2.5 nM PlaB.

FIG. 6: Spliceosome Perturbation induces necroptosis.

FIG. 7: Western Blot analysis showing pTBK1 protein depletion.

FIG. 8A-8B: Spliceosome Perturbation induces RIPK1-dependent apoptosis. (FIG. 8A) H82 and H446 cells were treated with PlaB, PlaB+Z-vad, PlaB+R3i, and PlaB+Z-Vad+R3i. H82 and H446 cells were treated with PlaB, PlaB+Z-vad, and PlaB+Necl.

FIG. 9A-9E: SF3B1-KO model data showing that spliceosome perturbation induces necroptosis. (FIG. 9A) Western blot showing SF3B1 KO in a hypertriploid human cell line (H446). (FIG. 9B) SF3B1KO induces accumulation of Z-NA in H446 cells. (FIG. 9C) Quantification of A-RNA and Z-NA in SF3B1 KO H446 cells and wt H446 cells. (FIG. 9D) SF3B1 knockout induces potent IFN response in SCLC cell line, H446. (FIG. 9E) Treatment of cells with HLA ABC and PD-L1 antibodies. H446 EV or EV refers to H446 cells treated with an empty vector.

FIG. 10: Spliceosome inhibition induces necroptosis in the Primary Lung Fibroblast of mouse model.

FIG. 11A-11B: Analysis of Z-NA and A-RNA sources. (FIG. 11A) A-Nucleic acid and A-Nucleic acid accumulation in MEF cells after treatment with PlaB with or without Dnasel, RnaseA, or Rnase H. (FIG. 11B) Quantification of A-RNA and Z-Nucleic acids in treated cells. MEF EV refers to MEF cells treated with an empty vector.

FIG. 12: Treatment of necroptosis competent SCLC model.

FIG. 13: Comparison of necroptosis in RPPM631 cells with and without SF3B1 knockout. RPPM631 EV or EV refers to RPPM631 cells treated with an empty vector.

FIG. 14: Analysis of ZBP1-dependent Necroptosis in RPPM631 cells with and without SF3B1 knockout. EV refers to RPPM631 cells treated with an empty vector.

FIG. 15A-15D: CytoToxicity analysis of PlaB in TNFR1-KO and TRADD-KO H82 cells (FIG. 16A) and H446 cells (FIG. 16B). CytoToxicity analysis of PlaB in FADD-KO and CASP8-KO H82 cells (FIG. 16C) and H446 cells (FIG. 16D). EV refers to cells treated with an empty vector.

FIG. 16: Analysis of the effect of spliceosome inhibition on necroptosis in WT MEF cells. EV refers to cells treated with an empty vector.

FIG. 17: Analysis of combination therapy in mouse model using PlaB and αPD-1.

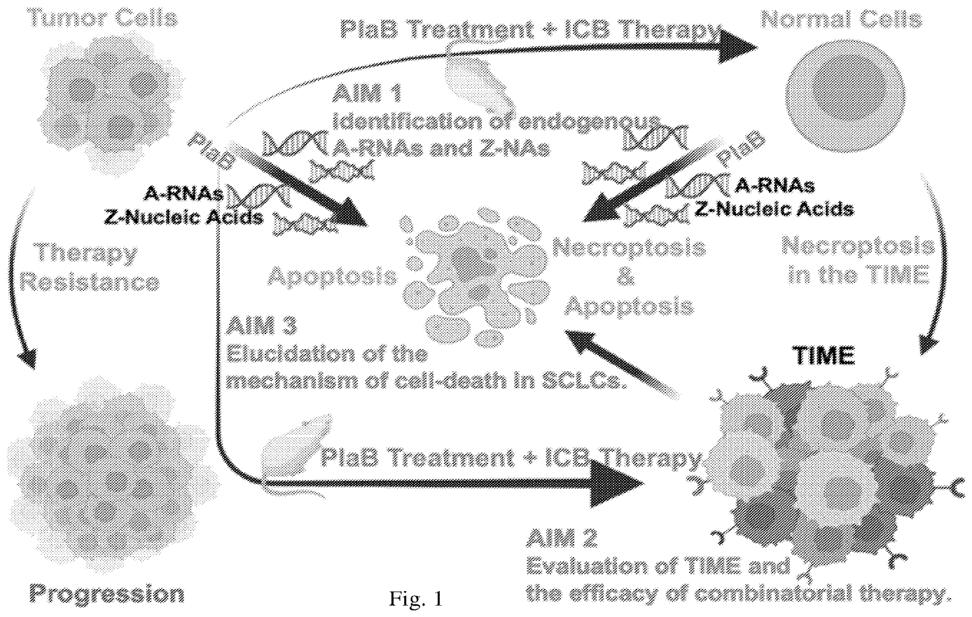

### DETAILED DESCRIPTION OF THE INVENTION

The spliccosome is a dynamic multiprotein complex that diversifies the transcriptome and proteome²⁶ and removes introns from precursor (pre-) mRNA to generate mature mRNA²⁷⁻²⁹. Large-scale sequencing studies have identified cancers that exhibit deregulated splicing producing irregular transcriptomes and proteomes associated with disease progression³⁰⁻³³. Some highly proliferative tumors with MYC hyper-activation have deregulated RNA repertoire and increased burden on splicing components resulting in spliceosome dependencies.

SF3B1, the largest subunit of the core spliceosome factor 3b (SF3b) complex, plays an integral part of RNA splicing fidelity^{28,38}, and is essential in tumors³⁹⁻⁴⁴. Studies show that splicing factor deregulation can result in loss of intron retention in tumors³⁰, and that intron retention is the mechanism for tumor-suppressor inactivation⁴⁵. Intron retention in cancers is dynamic⁴⁶, diversifies cancer transcriptome⁴³, establishes inter-and intra-tumoral heterogeneity^{47,48}, and enhances neoantigens to promote stemness and aggressiveness^{49,50}. Collectively, these studies demonstrate the significant role of splicing deregulation during tumor formation and progression.

Double-stranded (ds)RNA molecules (A-RNA) can also trigger an innate immune response. Pattern recognition receptors recognize these A-RNA and initiate production of type I interferons (IFNs) to activate antiviral responses⁵¹⁻⁵⁵. Viral mimicry is an antiviral response triggered by endogenous A-RNA, Z-NA, and transposable elements.

The data presented herein indicate that SCLCs respond favorably to spliceosome inhibition, especially inhibition targeting the splicing core component, SF3B1. The data demonstrate inhibition following treatment with small molecule inhibitors, such as Pladienolide B (PlaB). Additionally, we determined that spliceosome perturbation triggers a robust anti-tumor response through accumulation of double-stranded (ds)RNA (A-RNA) and, strikingly, the induction of uncommon left-handed Z-Nucleic acids (Z-NAs). These endogenous A-RNAs and Z-NAs together can initiate tumor-intrinsic antiviral interferon (IFN) signaling, and tumor-apoptosis. Furthermore, we found that spliceosome inhibition also induces necroptosis in necroptotic competent mouse embryonic fibroblast (MEF) through activation of Z-DNA Binding Protein 1 (ZBP1) by Z-NAs.

The data provided herein display three novel features. First, the data shows that spliceosome perturbation promotes robust viral mimicry responses through the induction of A-RNA and Z-NA in vitro in both SCLC cells and MEFs. Second, the data presented herein shows that spliccosome inhibition in SCLCs induces tumor-intrinsic immunogenicity, antigenicity, and PD-L1 expression that are known to play crucial roles in immune checkpoint blockade (ICB) therapies. Lastly, our data show that spliceosome inhibition potently induces cell death in SCLC cells.

### Definitions

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

The terms “about” or “approximately” in the context of numerical values and ranges refers to values or ranges that approximate or are close to the recited values or ranges such that the embodiment may perform as intended, such as having a desired amount of nucleic acids or polypeptides in a reaction mixture, as is apparent to the skilled person from the teachings contained herein. In some embodiments, about means plus or minus 10% of a numerical amount.

Furthermore, a compound “selected from the group consisting of” refers to one or more of the compounds in the list that follows, including mixtures (i.e., combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The terms “agent” and “test compound” denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid-based molecule which encoded the proteins described herein.

It is also contemplated that the term “compound” or “compounds” refers to the compounds discussed herein and includes precursors and derivatives of the compounds, and pharmaceutically acceptable salts of the compounds, precursors, and derivatives.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

The term “delivery” as used herein refers to the introduction of foreign molecule (i.e., miRNA encoding the polypeptide of interest) into cells. The term “administration” as used herein means the introduction of a foreign molecule into a cell. The term is intended to be synonymous with the term “delivery”.

### Spliceosome Inhibition

The spliceosome is a multi-megadalton complex of ribonucleoprotein (snRNP) particles, which are each composed of one or more uridine-rich small nuclear RNAs and several proteins. The snRNA components of the spliceosome promote the two transesterification reactions of splicing, among other functions. Initial RNA transcripts (pre-mRNA) of most eukaryotic genes are retained in the nucleus until non-coding intron sequences are removed by the spliceosome to produce mature messenger RNA (mRNA). The splicing that occurs can vary, so the synthesis of alternative protein products from the same primary transcript can be affected by tissue-specific or developmental signals. A significant fraction of human genetic diseases, including a number of cancers, are believed to result from deviations in the normal pattern of pre-mRNA splicing. As used herein, a “spliceosome” refers to this ribonucleoprotein complex that removes introns from one or more RNA segments, such as pre-mRNA segments.

As used herein, the terms “splicing modulator,” “spliceosome modulator,” or “splice modulator” refer to compounds that have anti-cancer activity by interacting with components of the spliceosome. In some embodiments, a splicing modulator alters the rate or form of splicing in a target cell. Splicing modulators that function as inhibitory agents, for example, are capable of decreasing uncontrolled cellular proliferation. Such modulators may be natural compounds or synthetic compounds. Non-limiting examples of splicing modulators and categories of such modulators include pladienolide (e.g., pladienolide D or pladienolide B), pladienolide derivatives (e.g., pladienolide D or pladienolide B derivatives), indisulam, indisulam derivatives, herboxidiene, herboxidiene derivatives, spliceostatin, spliceostatin derivatives, sudemycin, or sudemycin derivatives.

As used herein, the terms “derivative” and “analog” when referring to a splicing modulator, or the like, means any such compound that retains essentially the same, similar, or enhanced biological function or activity as the original compound but has an altered chemical or biological structure. In some embodiments, the splicing modulator is a pladienolide or pladienolide derivative.

As used herein, a “pladienolide derivative” refers to a compound which is structurally related to a member of the family of natural products known as the pladienolides and which retains one or more biological functions of the starting compound. Pladienolides were first identified in the bacteria Streptomyces platensis (Mizui et al. (2004) J Antibiot. 57:188-96) as being potently cytotoxic and resulting in cell cycle arrest in the G1 and G2/M phases of the cell cycle (e.g., Bonnal et al. (2012) Nat Rev Drug Dis 11:847-59). There are seven naturally occurring pladienolides, pladienolide A-G (Mizui et al. (2004) J Antibiot. 57:188-96; Sakai et al. (2004) J Antibiotics 57:180-7). U.S. Pat. Nos. 7,884,128 and 7,816,401 describe exemplary methods of synthesizing pladienolide B and D and are each incorporated herein by reference for such methods. Synthesis of pladienolide B and D may also be performed using the exemplary methods described in Kanada et al. ((2007) Angew Chem Int Ed. 46:4350-5). Kanada et al. and Intl. Pub. No. WO 2003/099813 describe exemplary methods for synthesizing E7107(D11) (Compound 45 of WO 2003/099813) from Pladienolide D (11107D of WO 2003/099813). A corresponding U.S. Pat. No. 7,550,503 to Kotake et al. Each of these references is incorporated herein for the described synthesis methods.

The terms “inhibition” or “inhibit” refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, “reduce” or “inhibit” refers to the ability to cause an overall decrease of 20% or greater. In another embodiment, “reduce” or “inhibit” refers to the ability to cause an overall decrease of 50% or greater. In yet another embodiment, “reduce” or “inhibit” refers to the ability to cause an overall decrease of 75%, 85%, 90%, 95%, or greater. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

The term “inhibitor” refers to an agent that slows down or prevents a particular chemical reaction, signaling pathway or other process, or that reduces the activity of a particular reactant, catalyst, or enzyme.

In some embodiments, the splicing modulators may act by binding to the SF3b spliceosome complex. Exemplary SF3b inhibitors include, without limitation, SF3B1 inhibitors, isoginkgetin, madrasin, tetrocarcin A, N-palmitoyl-L-leucine, psoromic acid, clotrimazole, NSC635326, and napthazarin. In certain embodiments, the splicing modulator acts by binding to SF3B1, the largest subunit of the core spliceosome factor 3b (SF3b) complex. The phrase “SF3B1 inhibitor” refers to a class of agents that inhibit the action of SF3B1. Exemplary SF3B1 inhibitors include, without limitation, pladienolide (e.g., pladienolide D or pladienolide B or pladienolides A-G), pladienolide derivatives (e.g., pladienolide D or pladienolide B derivatives), indisulam, indisulam derivatives, H3B-8800, H3B-8800 derivatives, FR901463, FR901464, FR901465, splicostatins A-G, thailanstatins A-C, meamycins, E7107, FD-895, herboxidiene, GEX1A, GEX1Q1-5, RON-18690A (18-deoxyherboxidiene), sudemycins C1, sudemycins D1, sudemycins F1, sudemycins E, and sudemycins D6.

In certain embodiments, the splicing modulators remodel the tumor-immune microenvironment (TIME). The phrase “tumor-immune microenvironment” or “TIME” refers to the normal cells, molecules, and blood vessels that surround and protect or attach to a tumor cell. A tumor can change its TIME and the TIME can affect how a tumor grows and spreads. These cells include lymphocytes with tumor suppressor effects, such as CD8+ T cells and natural killer cells, as well as some tumor-promoting cells with immunosuppressive functions, such as regulatory T cells and myeloid-derived suppressor cells. The spliceosome modulators of the present invention work to remodel the TIME to enhance tumor suppression, cell apoptosis, and cell necroptosis.

The term “apoptosis”, or “programmed cellular death”, refers to an active process of cell death. Typically, the process requires ATP, involves new RNA and protein synthesis, and culminates in the activation of endogenous endonucleases that degrade the DNA of the cell, thereby destroying the genetic template required for cellular hemostasis. Apoptosis is observed in controlled deletion of cells during metamorphosis, differentiation, and general cell turnover and appears normally to be regulated by receptor-coupled events. For these reasons, apoptosis has been called “programmed cell death” or “cell suicide.” While every cell likely has the genetic program to commit suicide, it is usually suppressed. Under normal circumstances, only those cells no longer required by the organism activate this self-destruction program.

Apoptotic cell death is characterized by plasma membrane blebbing, cell volume loss, nuclear condensation, and endonucleolytic degradation of DNA at nucleosome intervals. Loss of plasma membrane integrity is a relatively late event in apoptosis, unlike the form of cell death termed necrosis, which can be caused by hypoxia and exposure to certain toxins and which is typically characterized early-on by increased membrane permeability and cell rupture.

“Necroptosis,” as used herein, refers to a regulated, caspase-independent cell death, that can be an alternative way to eliminate apoptosis-resistant cancer cells. The core necroptotic pathway consisting of a receptor-interacting protein kinase 1 (RIP1 or RIPK1)—receptor-interacting protein kinase 3 (RIP3 or RIPK3)—mixed lineage kinase domain-like protein (MLKL) complex, also called the ‘necrosome’. The necrosome initiates downstream effector functions such as generation of a reactive oxygen species (ROS) burst, plasma membrane permeabilization, and cytosolic ATP reduction that further drives irreversible necroptosis-executing mechanisms. Provided herein are methods of treating patients by modulating necroptosis comprising administering to a patient a disclosed antisense oligonucleotide.

In certain embodiments, the spliceosome modulators increase concentrations of A-RNA and Z-Nucleic acids. The structure of double-stranded DNA (dsDNA) in nature can be broadly categorized into 3 major forms, namely compact right-handed A-DNA, loose right-handed B-DNA and the unique left-handed Z-DNA conformation. Unlike the anti-conformation base arrangement throughout A-and B-DNA, the nucleoside bases in Z-DNA adopt alternating syn-and anti-conformation bases, giving rise to its distinctive left-handed double helical structure with zigzag backbone (thus its name). Similar to dsDNA, double-stranded RNA (dsRNA) can also adopt the Z-conformation. Under normal physiological conditions, Z-DNA/Z-RNA exist at higher energy configuration and thus are energetically unstable on their own.

Z-DNA-binding protein 1 (ZBP1), alternatively known as DNA-dependent activator of IFN-regulatory factors (DAI) or Tumor stroma and activated macrophage protein (DLM1), plays a significant role in innate immune response against viruses or other non-self-agents. Activation of ZBP1 then leads to downstream signal transduction mediated through the RHIM domain interactions with receptor-interacting protein (RIP) kinases, regulating apoptosis, inflammation, and interferon responses to pathogens. In addition, ZBP1 sensing activates NLRP3 inflammasome complex that leads to PAN-optosis (pyroptosis, apoptosis, and necroptosis) process.

### Methods of Treatment and Administration

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, or stabilize, a pathological condition or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. It is understood that treatment, while intended to cure, ameliorate, or stabilize, a disease, pathological condition, or disorder, need not actually result in the cure, ameliorization, or stabilization. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

As used herein, the terms “tumor”, “tumor growth” or “tumor tissue” can be used interchangeably, and refer to an abnormal growth of tissue resulting from uncontrolled progressive multiplication of cells and serving no physiological function. A solid tumor can be malignant, e.g. tending to metastasize and being life threatening, or benign. Examples of solid tumors that can be treated or prevented according to a method of the present invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastic cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, glioblastoma, and retinoblastoma.

The phrase “cold cancer” refers to a cancer or tumor in a patient categorized by low immune infiltrates and evasion from the host immune responses. The phrase “tumor mutational burden” or “TMB” refers to the number of genetic changes or mutations in a cancer cell. The immune system can identify cancer cells and activate an immune response by detecting these mutations. Accordingly, cancers with low TMB have fewer mutations and a decreased chance of activating the immune system. Conversely, cancers with high TMB have more mutations and an increased chance of activating the immune system. Treatment of these difficult to treat cancers is considered herein.

Cancer treatments which may be used in combination with the spliceosome modulators provided herein include, without limitation, surgery, chemotherapy, radiation therapy, targeted therapy, immunotherapy, and/or hormone therapy. Known chemotherapeutic agents include, without limitation alkylating agent, anti-metabolic antineoplastic agent, anti-tumor antibiotic, anti-tumor botanical, platinum compound antineoplastic agent, hormonal balance antineoplastic agent, and miscellaneous antineoplastic agent, wherein therapeutical agent used in said targeted therapy is selected from the group consisting of rituximab, bevacizumab, trastuzumab, imatinib, dinoxetine, cetuximab, nilotinib, and sorafenib, wherein therapeutical agent used in said immunotherapy is selected from the group consisting of PD-1 inhibitor, PD-L1 inhibitor and CTLA4 inhibitor; more preferably, said alkylating agent is selected from the group consisting of cyclophosphamide, ifosfamide and thiotepa, said anti-metabolic antineoplastic agent is selected from the group consisting of methotrexate, mercaptopurine, fluorouracil and cytarabine, said anti-tumor antibiotic is selected from the group consisting of bleomycin, daunorubicin, actinomycin D, mitomycin, doxorubicin and mitoxantrone, said anti-tumor botanical is selected from the group consisting of vincristine, etoposide, teniposide, paclitaxel and docetaxel, said platinum compound antineoplastic agent is selected from the group consisting of cisplatin, carboplatin and oxaliplatin, said hormone balance antineoplastic agent is selected from the group consisting of leuprolide, tamoxifen, flutamide and formestane, said miscellaneous antineoplastic agent is arsenic trioxide.

The term “drug response” as used herein, means any biological response in an organism that is the result of exposure to the drug. Drug responses can be favorable, such as when a patient's disease is eradicated by treatment with the drug, or unfavorable, such as when a patient enters a coma upon treatment with a drug.

The term “synergy” or “synergistic” refers to the interaction or cooperation of two or more substances, or other agents to produce a combined effect greater than the sum of their separate effects.

In certain embodiments, treatment using two or more substances improves the therapy using either one of the substances alone, by maximizing efficacy, reducing toxicity, and addressing interpatient variability, as well as delaying and/or overcoming innate or acquired resistance.

Provided herein are methods of treatment of cancer including lung cancers. In certain embodiments, the cancer is a COLD cancer or a cancer with a low tumor mutated burden (TMB). In certain embodiments, the cancer is small cell lung cancer (SCLC). The methods include administration of an effective amount of at least one spliceosome modulator to a subject in need thereof. In certain embodiments, the spliceosome modulator is selected from a small molecule inhibitor, such as SF3B1 inhibitors, anti-SF3B1 antibodies, nucleotide aptamers, soluble receptors, or other compounds. SF3B1 inhibitors include without limitation pladienolide (e.g., pladienolide D or pladienolide B), pladienolide derivatives (e.g., pladienolide D or pladienolide B derivatives), herboxidiene, herboxidiene derivatives, spliceostatin, spliceostatin derivatives, sudemycin, or sudemycin derivatives. In some embodiments, the symptoms of the cancer are reduced, as compared to a control.

In certain embodiments, the methods include administration of an additional chemotherapeutic agent or chemotherapy. In certain embodiments, the additional therapy is an immune checkpoint blockade therapy, such as anti-PD-L1 antibodies. In certain embodiment the additional therapy is surgery, chemotherapy, radiation therapy, targeted therapy, immunotherapy, and/or hormone therapy. In certain embodiments, the chemotherapeutic agents is alkylating agent, anti-metabolic antineoplastic agent, anti-tumor antibiotic, anti-tumor botanical, platinum compound antineoplastic agent, hormonal balance antineoplastic agent, and miscellaneous antineoplastic agent, wherein therapeutical agent used in said targeted therapy is selected from the group consisting of rituximab, bevacizumab, trastuzumab, imatinib, dinoxetine, cetuximab, nilotinib, and sorafenib, wherein therapeutical agent used in said immunotherapy is selected from the group consisting of PD-1 inhibitor, PD-L1 inhibitor and CTLA4 inhibitor; more preferably, said alkylating agent is selected from the group consisting of cyclophosphamide, ifosfamide and thiotepa, said anti-metabolic antineoplastic agent is selected from the group consisting of methotrexate, mercaptopurine, fluorouracil and cytarabine, said anti-tumor antibiotic is selected from the group consisting of bleomycin, daunorubicin, actinomycin D, mitomycin, doxorubicin and mitoxantrone, said anti-tumor botanical is selected from the group consisting of vincristine, etoposide, teniposide, paclitaxel and docetaxel, said platinum compound antineoplastic agent is selected from the group consisting of cisplatin, carboplatin and oxaliplatin, said hormone balance antineoplastic agent is selected from the group consisting of leuprolide, tamoxifen, flutamide and formestane, said miscellaneous antineoplastic agent is arsenic trioxide.

In certain embodiments, the method of treatment effectively suppresses symptoms associated with cancer. Symptoms of vary according to the location and type of cancer being treated. In certain embodiments, symptoms of cancer include, fatigue, weight loss, lumps, pain coughing, wheezing, new or unusual growth, discoloration, and no symptoms at all. In certain embodiments, the treatment reduces the risk of relapse. In the context of a cancer, treatment or inhibition may be assessed by inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS, as used herein means the time from treatment onset until death from any cause. TTP, as used herein refers to the time from treatment onset until tumor progression; TTP does not include deaths. Time to Remission (TTR) as used herein means the time from treatment onset until remission, for example, complete or partial remission. As used herein, PFS means the time from treatment onset until tumor progression or death. In one embodiment, PFS rates will be computed using the Kaplan-Meier estimates. Event-free survival (EFS) means the time from study entry until any treatment failure, including disease progression, treatment discontinuation for any reason, or death. Relapse-free survival (RFS) means the length of time after the treatment ends that the patient survives without any signs or symptoms of that cancer. Overall response rate (ORR) means the sum of the percentage of patients who achieve complete and partial responses. Complete remission rate (CRR) refers to the percentage of patients achieving complete remission (CR). Duration of response (DoR) is the time from achieving a response until relapse or disease progression. Duration of remission is the time from achieving remission, for example, complete or partial remission, until relapse. In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

The compounds described herein can be formulated for enteral, parenteral, topical, or systemic administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. Typical carriers and conventional methods of preparing pharmaceutical compositions that can be used in conjunction with the preparation of formulations of the compounds are known by those skilled in the art. These compositions can take the form of solutions, suspensions, emulsion, tablets, pills, capsules, powders, sustained-release formulations and the like.

The compounds described herein can be formulated for parenteral administration. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

For intravenous administration, the compositions may be packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thercof.

Suitable surfactants may be anionic, cationic, amphoteric or nonionic surface-active agents. Suitable anionic surfactants include, but are not limited to, those containing carboxylate, sulfonate and sulfate ions.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above.

The compounds described herein can be administered in an effective amount to a subject that is in need of alleviation or amelioration from one or more symptoms associated with tumor growth.

The exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation. The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The compositions are administered in an effective amount and for a period of time effect to reduce one or more symptoms associated with the disease to be treated. It should be understood that the “effective amount” for a composition having anti-cancer cell proliferation properties may vary. In one embodiment an effective amount includes without limitation about 0.001 to about 25 mg/kg subject body weight. In one embodiment, the range of effective amount is 0.001 to 0.01 mg/kg body weight. In another embodiment, the range of effective amount is 0.001 to 0.1 mg/kg body weight. In another embodiment, the range of effective amount is 0.001 to 1 mg/kg body weight. In another embodiment, the range of effective amount is 0.001 to 10 mg/kg body weight. In another embodiment, the range of effective amount is 0.001 to 20 mg/kg body weight. In another embodiment, the range of effective amount is 0.01 to 25 mg/kg body weight. In another embodiment, the range of effective amount is 0.01 to 0.1 mg/kg body weight. In another embodiment, the range of effective amount is 0.01 to 1 mg/kg body weight. In another embodiment, the range of effective amount is 0.01 to 10 mg/kg body weight. In another embodiment, the range of effective amount is 0.01 to 20 mg/kg body weight. In another embodiment, the range of effective amount is 0.1 to 25 mg/kg body weight. In another embodiment, the range of effective amount is 0.1 to 1 mg/kg body weight. In another embodiment, the range of effective amount is 0.1 to 10 mg/kg body weight. In another embodiment, the range of effective amount is 0.1 to 20 mg/kg body weight. In another embodiment, the range of effective amount is 1 to 25 mg/kg body weight. In another embodiment, the range of effective amount is 1 to 5 mg/kg body weight. In another embodiment, the range of effective amount is 1 to 10 mg/kg body weight. In another embodiment, the range of effective amount is 10 to 20 mg/kg body weight. In another embodiment, the range of effective amount is 20 to 30 mg/kg body weight. In another embodiment, the range of effective amount is 30 to 40 mg/kg body weight. In another embodiment, the range of effective amount is 40 to 50 mg/kg body weight. In another embodiment, the range of effective amount is 1 to 50 mg/kg body weight. Still other doses falling within these ranges are expected to be useful.

In another embodiment, the range of effective amount is 0.001 mg to 10 g. In another embodiment, the range of effective amount is 0.01 mg to 1 g. In another embodiment, the range of effective amount is 0.01 mg to 100 mg. In another embodiment, the range of effective amount is 0.1 mg to 100 mg. In another embodiment, the range of effective amount is 0.1 mg to 500 mg. In another embodiment, the range of effective amount is 1 mg to 100 mg. In another embodiment, the range of effective amount is 10 mg to 500 mg. In another embodiment, the range of effective amount is 10 mg to 750 mg. In another embodiment, the range of effective amount is 0.01 mg to 100 mg. In another embodiment, the range of effective amount is 1 mg to 500 mg.

In certain embodiments, the spliceosome inhibitor is administered via intertumoral administration. In these embodiments, the effective amount of the spliceosome inhibitor may be between 0-200 nM, 0-150 nM, 0-100 nM, 0-50 nM, 0-25 nM, 25 nm-200 nM, 25-150 nM, 25-100 nM, 25-50 nM, 50 nM-200 nM, 50-150 nM, 50-100 nM, 100-200 nM, 100-150 nM, 150 nM-200 nM, about 25 nM, about 50 nM, about 100 nM, about 150 nM or about 200 nM. In certain embodiments PlaB is administered via intratumural administration at any one of the doses above.

In certain embodiments, the spliceosome inhibitor is administered via intraperitoneal administration. In these embodiments, the effective amount of the spliceosome inhibitor may be between 2-20 mg/kg, 0-20 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg. In certain embodiments PlaB is administered via intraperitoneal administration at anyone of the doses above.

In certain embodiments, the spliceosome inhibitor is administered via systemic or intravenous administration. In these embodiments, the effective amount of the spliceosome inhibitor may be between 2-10 mg/kg, 2-20 mg/kg, 0-20 mg/kg, 14-50 mg/kg, 12.5-100 mg/kg, or at least about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In certain embodiments, PlaB is administered via systemic or intravenous administration at any one of the doses above. In certain embodiments, PlaB is administered at a dose of 2-20 mg/kg. In certain embodiments, sudemycin D6 is administered via systemic or intravenous administration at anyone of the doses above. In certain embodiments, sudemycin D6 is administered at a dose of 14 mg/kg-50 mg/kg. In certain embodiments, indisulam is administered via systemic or intravenous administration at anyone of the doses above. In certain embodiments, indisulam is administered at a dose of 12.5 mg/kg-100 mg/kg.

In certain embodiments, the spliceosome inhibitor is administered via oral administration. In these embodiments, the effective amount of the spliceosome inhibitor may be between 2-10 mg/kg, 2-20 mg/kg, 0-20 mg/kg, 0-40 mg/kg, 2-40 mg/kg, 10-40 mg/kg, 10-20 mg/kg, 20-40 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, or about 40 mg/kg. In certain embodiments H3B-8800 is administered via oral administration at any one of the doses above.

In certain embodiments, the combination therapies above are effective to reduce the effective amount of at least one of the spliceosome inhibitor, and the second therapy. In certain embodiments, the effective amount of the spliceosome inhibitor is reduced by 75%, 85%, 90%, 95%, or greater when compared to solo treatment. The effective amount can be reduced by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

In certain embodiments, the effective amount of the secondary therapy, such as an immunotherapy, is reduced by 75%, 85%, 90%, 95%, or greater when compared to solo treatment. The effective amount can be reduced by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

### Kits and Articles of Manufacture

Any of the aforementioned products can be incorporated into a kit which may contain a spliceosome modulator, a pharmaceutically acceptable carrier, instructions for use, a container, a vessel for administration, or any combination thereof.

The following examples are provided to illustrate certain embodiments of the invention. They are not intended to limit the invention in any way.

These Examples provide novel insights into how spliceosome modulation confer anti-tumor response to remodel the tumor-immune microenvironment (TIME) and elucidate an unexplored mechanism of aberrant splicing triggering ZBP1-dependent necroptosis and ZBP1-independent apoptosis to re-activate host anti-tumor immunity to combat SCLC malignancy. The Examples characterize novel A-RNA and Z-NA species from perturbed splicing and determine their genomic locations to better understand regions of the genome harboring potent immunogenic signals. Previously, ICB therapy could only minimally extend the overall survival in SCLC. However, the findings disclosed herein describe how spliceosome-targeted therapies and combinatorial therapies, such as spliceosome modulation with immunotherapy provide a more efficacious and personalized treatment for SCLC patients.

In addition, the findings show that spliceosome perturbation induces necroptosis in the TIME potently enough to activate anti-tumor response. Furthermore, the data elucidate the mechanism of spliceosome inhibition induced cell death which can be used to advantage to identify RNA biomarkers, create RNA therapeutics, and design combinatorial therapies for SCLC patients.

The Examples use innovative technical approaches involving the use of RNA immunoprecipitation (RIP) followed by total RNA-sequencing (RNA-Seq) to explore global immune-modulatory A-RNA and Z-NA species induced by SF3B1 inhibition. In addition, the combinatorial therapy (spliceosome inhibition and ICB therapy) is used to reveal tumor-immune interplay, elucidate TIME remodeling, assess synergy between ICB therapy and spliceosome modulation, and test whether necroptosis is induced in vivo.

The experiments performed herein were designed to identify splicing perturbation induced immunogenic A-RNA and Z-NA species that are targets of ZBP1, determine if spliceosome perturbation synergizes with ICB therapy, and further clarify the mechanism of SF3B1 inhibition induced cell death in necroptosis incompetent tumors. The data presented herein is further utilized for the development of targeted and combinatorial therapies to enhance anti-tumor immunity in SCLCs or other immunologically “COLD” tumors. The central hypothesis of the application is that spliceosome inhibition can induce immunogenic A-RNA and Z-NA species in both cancer cells and normal cells to induce a more immunogenic tumor-immune microenvironment (TIME) and ultimately improve the response to cancer immunotherapies.

These Examples not only provide insights into fundamentally important biology about spliceosome perturbation on host-tumor immunity and cell death, but also help to identify novel combinatorial therapies to enhance anti-tumor immunity and boost cancer immunotherapy in ‘COLD’ tumors, such as SCLC.

The following examples are provided to illustrate certain embodiments of the invention. They are not intended to limit the invention in any way.

### Example I

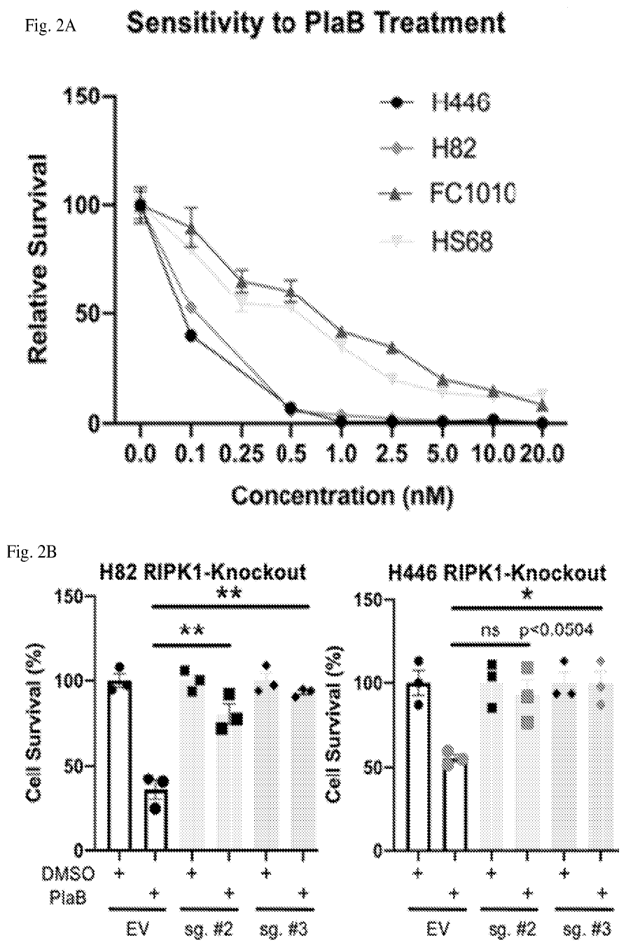

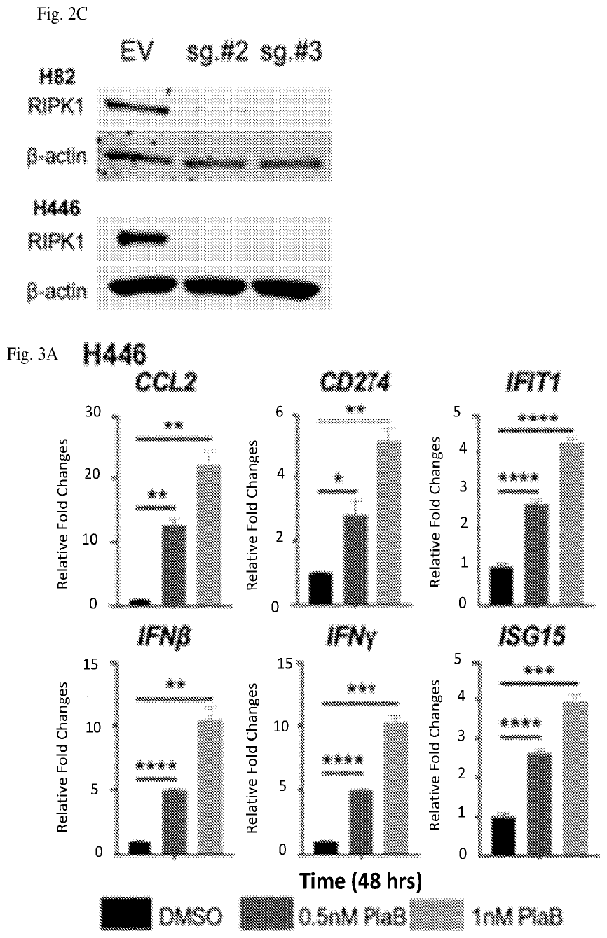

Cancer deregulates splicing^{31,32}, and we hypothesized that hyper-proliferative SCLCs are dependent on splicing machineries for increased tumorigenicity⁹⁰. We determined that SCLC cell lines, H446 and H82, are particularly sensitive to SF3B1 inhibition using nanomolar concentration of PlaB (FIG. 2A), which is rescued by RIPK1 depletion (FIGS. 2B and 2C).

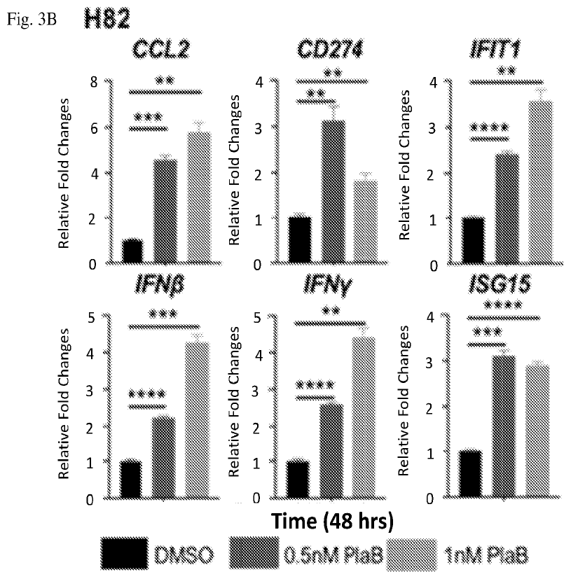

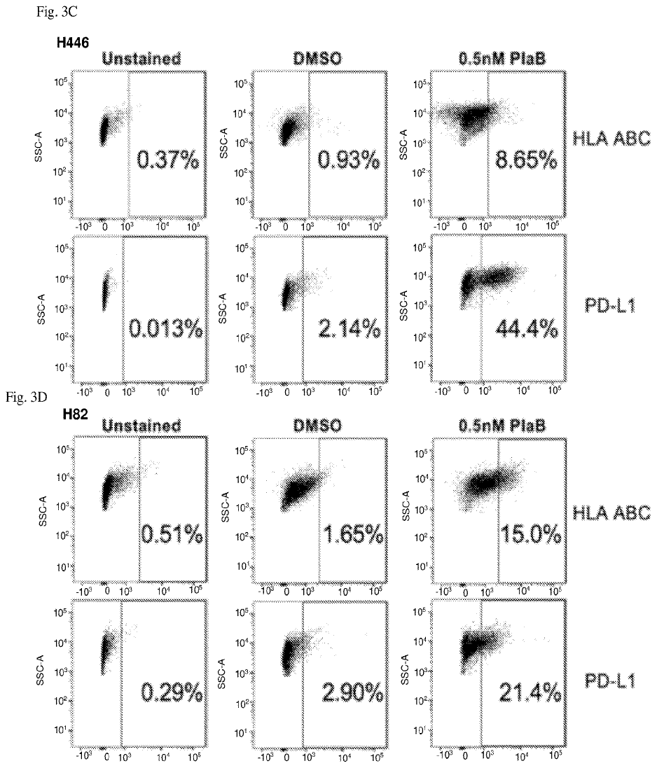

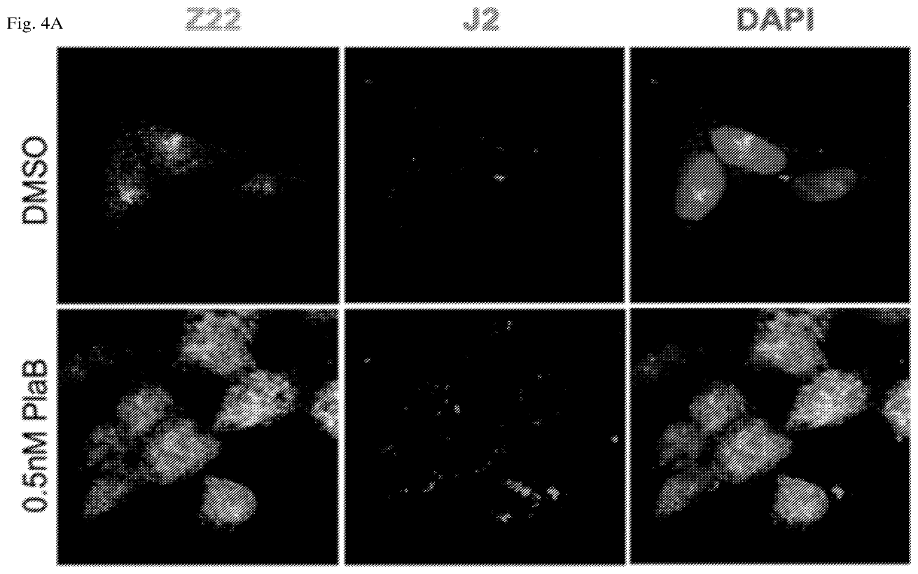

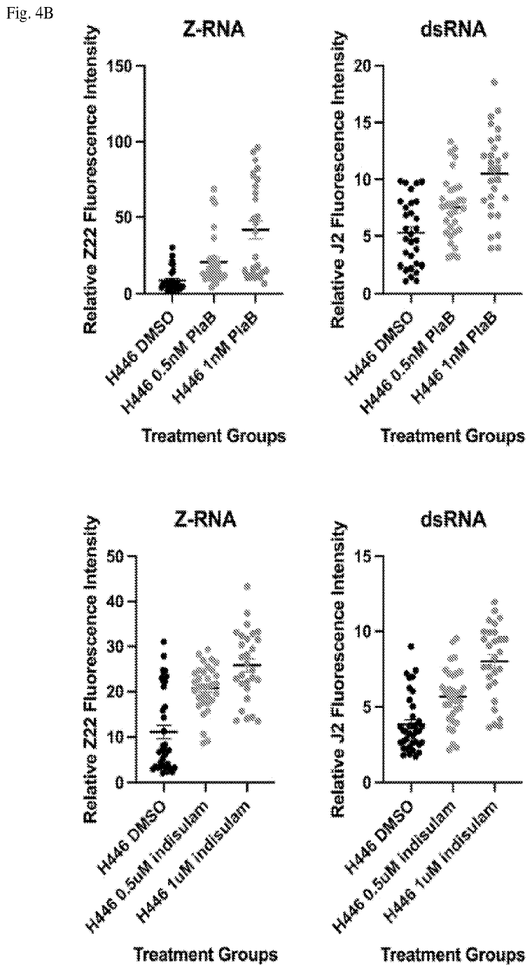

We also observed that SF3B1 inhibition triggers robust anti-tumor response and upregulates IFN-β, IFN-γ, and IFN-responsive genes by real-time reverse-transcription quantitative polymerase chain reaction (rRT-qPCR) in H446 and H82 cells (FIGS. 3A and 3B). These results led us to explore whether the robust IFN response in H82 and H446 cell lines are sufficient to alter tumor-intrinsic immunogenicity. We found that amplified IFN response strongly increased human leukocyte antigens ABC (HLA-ABC) and program death-ligand 1 (PD-L1) in H446 and H82 which indicates an effective ICB outcome (FIGS. 3C and 3D). These observations indicate that spliceosome inhibition is useful as a targeted therapy for SCLCs.

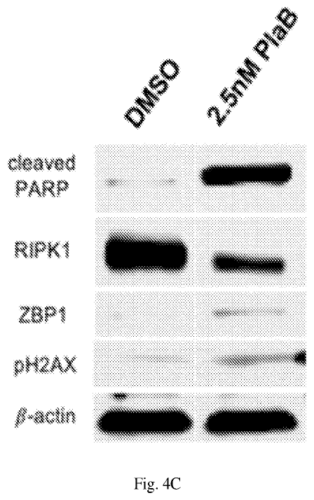

Our observations led us to explore what caused the elevated IFN response. We used an immunofluorescence (IF) technique to determine if A-RNA was increased after PlaB treatment. Surprisingly, after 6 hours of PlaB treatment, we not only found that there is a robust induction of A-RNA using mouse J2 antibody, but also a significant increase of uncommon immunogenic left-handed nucleic acids (Z-Nas) detected with rabbit Z22 antibody in H446 cells (FIGS. 4A and 4B).

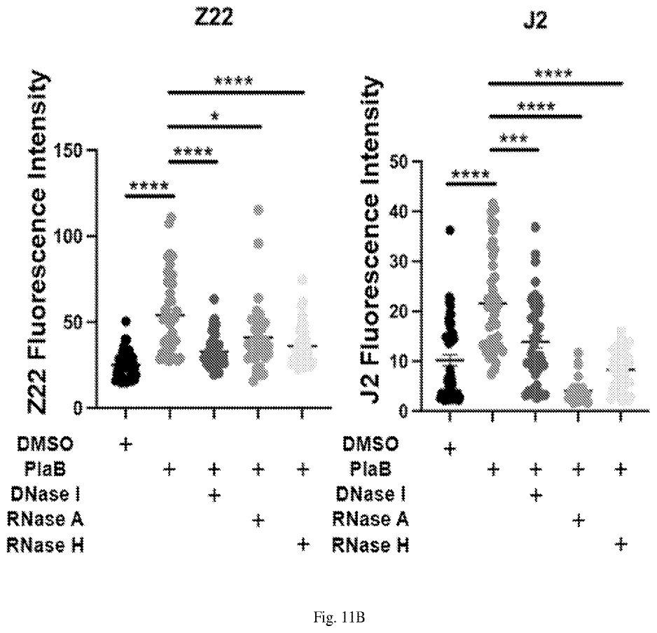

The increases in A-RNA and Z-NA was further analyzed in MEF cells. Cells were treated with PlaB alone, or in combination with dNasel (dsDNA), rNaseA(dsRNA at [high]), or dNaseH (DNA/RNA hybrid). After treatment, cells treated with PlaB had an increase in both A-RNAs and Z-nAs. Cells treated with PlaB and dNasel, rNaseA, or RnaseH still showed an increase in A-RNAs and Z-nAs, however, the increase in A-RNAs was not as robust as PlaB alone. (FIG. 11A-11B).

Furthermore, our western blot analysis showed increased cleaved-PARP, ZBP1, and pH2AX proteins after 48 hours of PlaB treatment. Unexpectedly, we determined that RIPK1 protein level dropped considerably (FIG. 4C). These results indicate that spliceosome perturbation triggers robust anti-tumor response through accumulation of A-RNA and Z-NA leading to intrinsic antiviral signaling and cell death in SCLC cell lines.

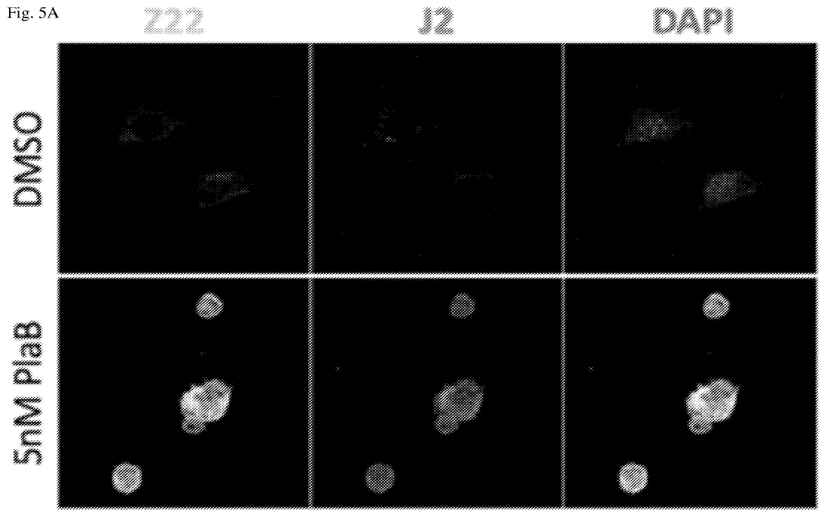

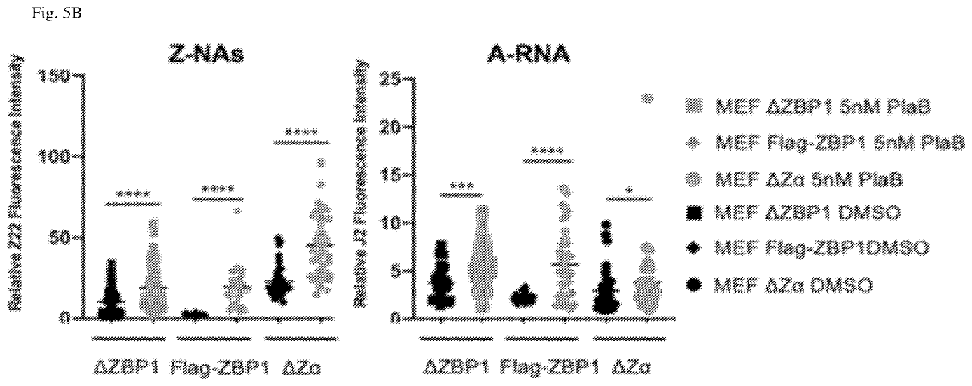

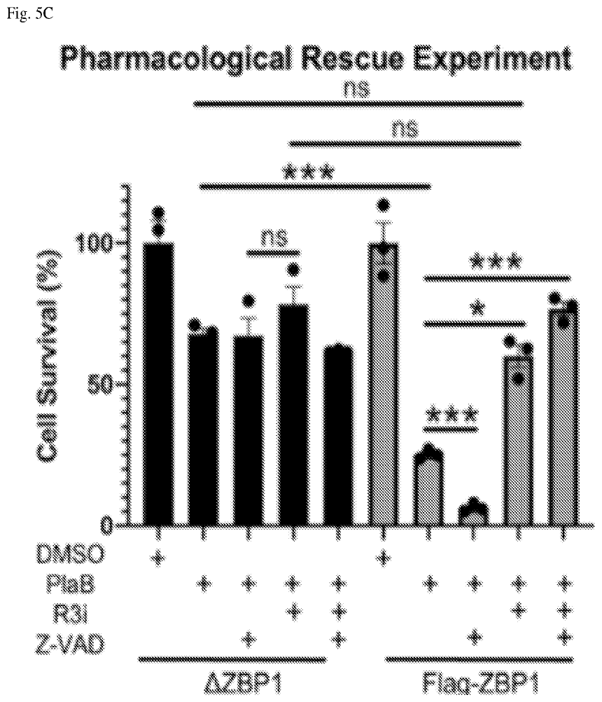

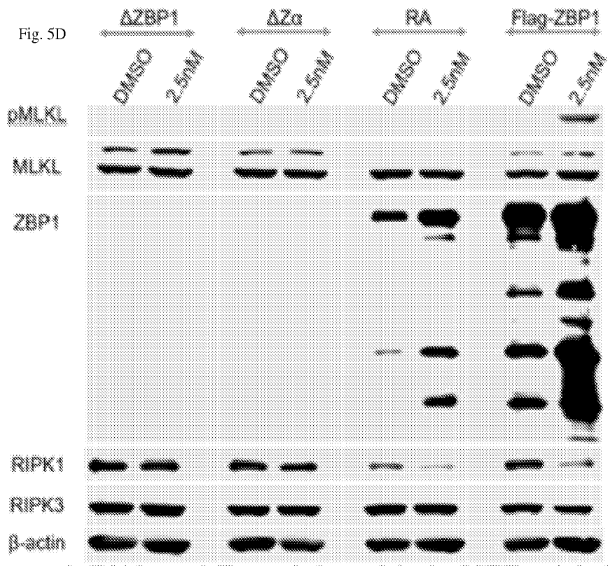

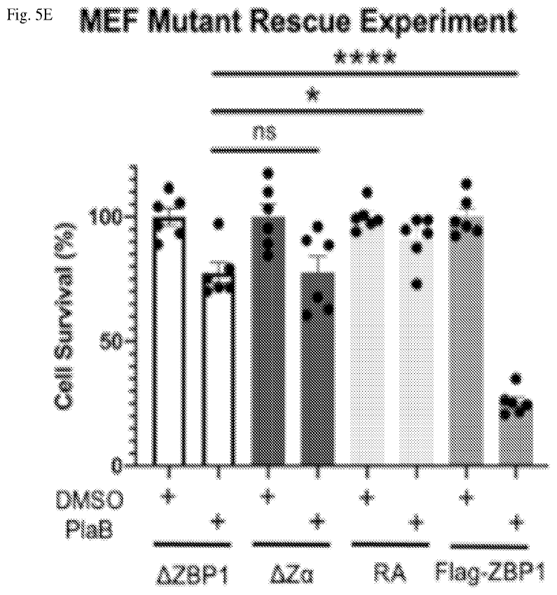

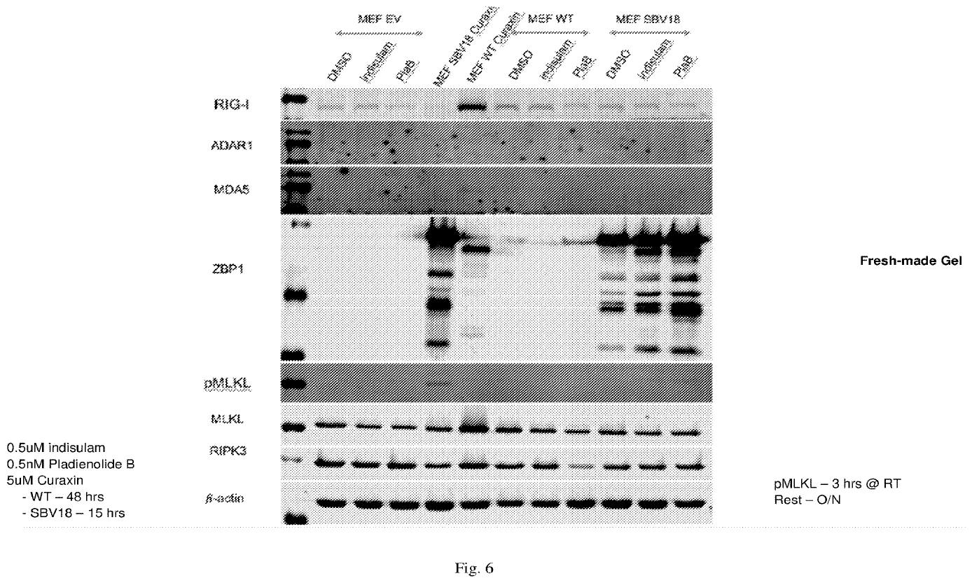

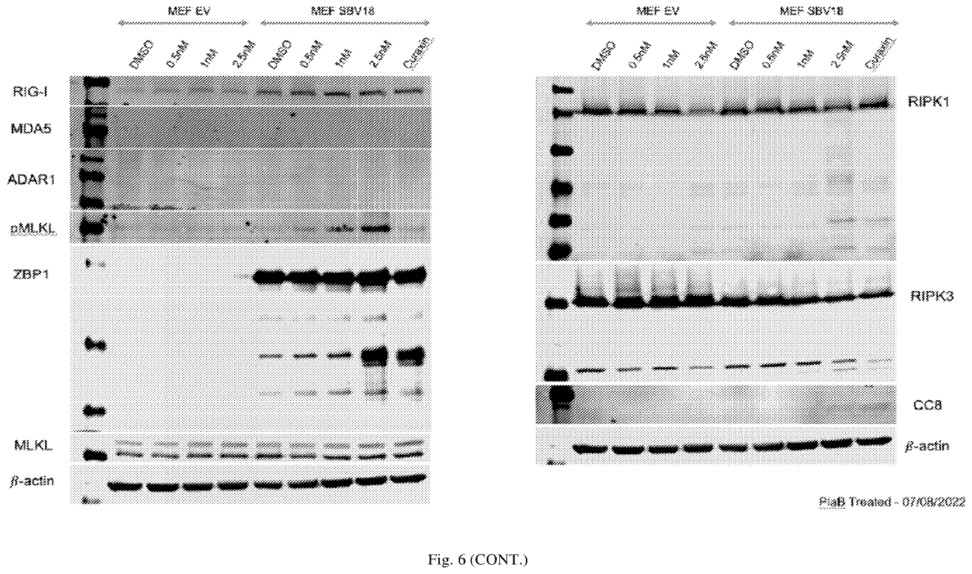

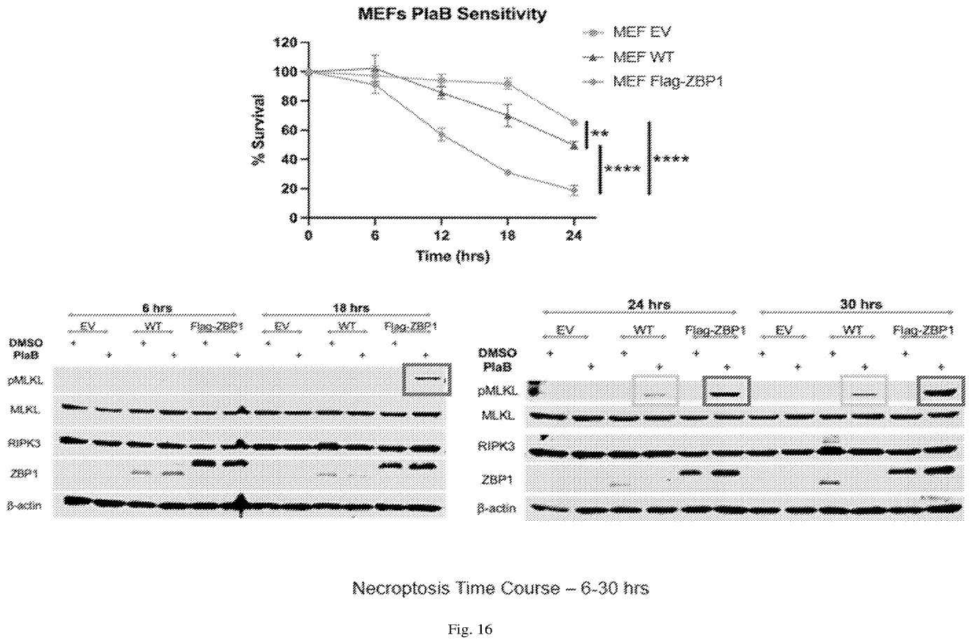

Since we observed a slight increase in ZBP1 protein level (FIG. 4C), we wondered if these accumulated Z-nAs can induce necroptosis. In a brief analysis of the CCLE mRNA expression in lung tumors, we observed that necroptosis pathways are downregulated (FIG. 6). Since induction of Z-nAs can result in ZBP1-dependent necroptosis, we analyzed induction of necroptosis in necroptotic competent MEFs using spliceosome inhibition. First, we found that SF3B1 inhibition resulted in accumulation of A-RNA and Z-NA in MEFs ΔZBP1, ΔZα, and Flag-ZBP1 (FIGS. 5A and 5B). We then determined that PlaB-mediated cell death was completely rescued by RIPK3 inhibitor GSK-843, R3i (FIG. 5C). Furthermore, spliccosome inhibition triggered pMLKL (necroptosis) only in MEF Flag-ZBP1 cells. However, western blot analysis showed that pMLKL was not triggered in MEFs ΔZBP1 and MEF, ΔZα, and MEF RA, which ultimately induces membrane rupture and immunogenic cell death (FIGS. 5D and 5E). We also determined that MEFs ΔZBP1, WT, and Flag-ZBP1 cells treated with PlaB began dying within 24hours after treatment. With MEF Flag-ZBP1 cells showing only 20% survival after 24 hours (FIG. 16).

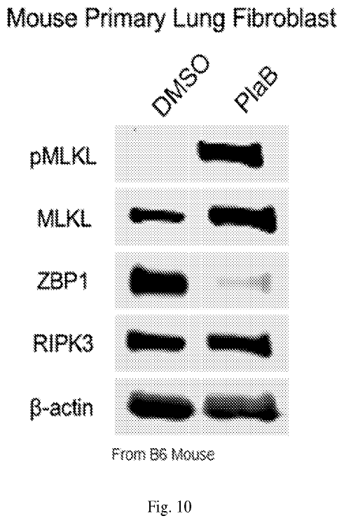

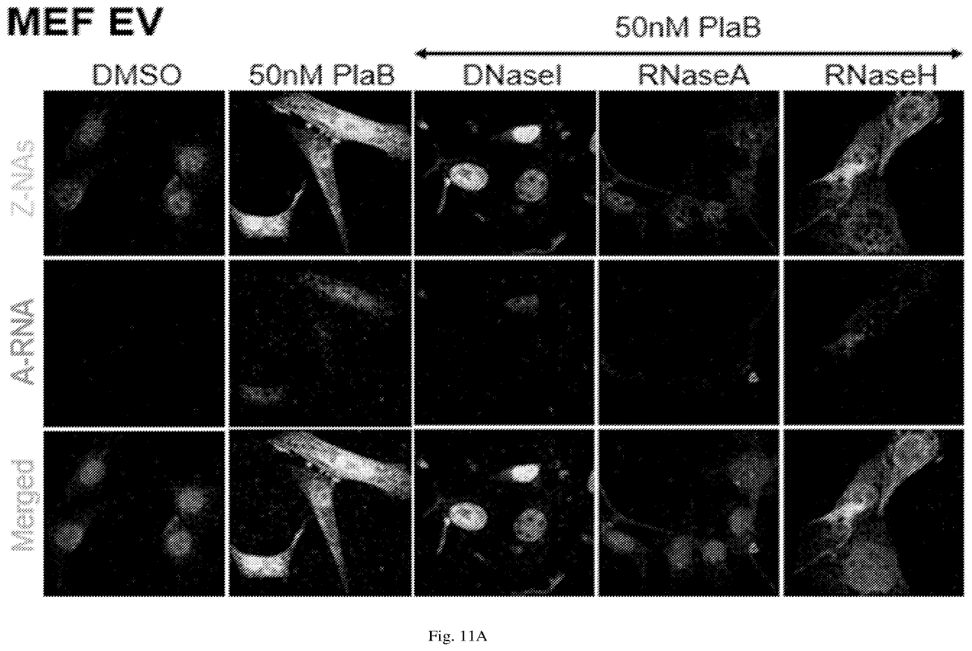

Further analysis of necroptosis in primary lung fibroblasts showed that spliceosome inhibition also triggered pMLKL (necroptosis) in the primary lung fibroblast. (FIG. 10)

These results indicate that spliceosome perturbation can induce apoptosis in SCLC cell lines, and induce immunogenic cell death and necroptosis, in fibroblasts. Altogether, our data presented in FIG. 2 through FIG. 5 and FIG. 10 show that spliceosome perturbation is a novel approach to concurrently induce endogenous Z-NA and A-RNA to trigger immunogenic cell death. Furthermore, these data indicate that spliceosome integrity is also the gatekeeper for endogenous Z-NAs activation.

### Example II: Characterization of Spliceosome Inhibition Induced Endogenous A-RNAs and Z-nAs in SCLC Cells and MEFs

Splicing is important for cell homeostasis²⁷⁻²⁹, but no studies have shown the link between perturbed splicing and Z-NA formation. Our data show that spliceosome inhibition in SCLC cell lines and MEFs leads to accumulation of A-RNA and Z-NA (FIGS. 4 and 5). Furthermore, these endogenous nucleic acids can induce potent immunogenic responses causing apoptosis and necroptosis⁸⁰⁻⁸² (FIGS. 2, 3, and 5). The identities of these immunogenic A-RNA and Z-NA remains unknown, and it is of great importance to identify these transcripts that can activate immunogenicity upon spliceosome inhibition.

To identify A-RNA and Z-NA species accumulated upon spliceosome perturbation, MEF ΔZBP1 cells and PlaB are utilized for the following experiments. MEF ΔZBP1 cells are divided into two experimental conditions in the absence and presence of PlaB, and then subsequently further divided into six groups: DMSO isotype control (immunoglobulin (Ig)G), DMSO J2, DMSO Z22, PlaB IgG, PlaB J2, and PlaB Z22. All conditions and doses of reagents used are the same as previously described in FIG. 5A. To characterize these immunogenic nucleic acid species, RIP-Scq is performed using J2 and Z22 antibodies to capture all native A-RNA and Z-NA species regardless of polyadenylation status to allow unbiased interrogation of immunogenic species that are degraded, immature, coding, and noncoding.

Furthermore, to capture native RNA species in vitro and minimize artifacts, cells are gently lysed with RIP buffer. For each condition, input and J2 enriched versus IgG RNAs are isolated. To further minimize RNA contamination (especially ribosomal (r)RNA which can comprise greater than 85% of the total RNA⁹¹, and has higher conserved secondary structures⁹²), all samples undergo rRNA depletion using commercially available rRNA removal kit to allow for precise interrogation of less abundant A-RNA and Z-NA transcripts. After RNA purification, samples are sent to Fox Chase Cancer Center (FCCC) Genomic Core for downstream processes, such as cDNA library preparation using Illumina TruSeq Stranded Total RNA kit and paired-end 150 cycle sequencing with a minimum of 40 million reads per sample for a robust downstream analysis. Sequencing results are further analyzed by FCCC Bioinformatic Facility to delineate A-RNA and Z-NA species that are upregulated by spliceosome inhibition.

RNA species that are enriched in IgG control are removed from each individual condition to precisely seek those targets in the presence of PlaB that have potent immunogenic properties that can be exploited therapeutically and future vaccine development to increase treatments for metastatic SCLCs. To confirm that the enriched immunogenic species identified here are universal across cell lines, RIP followed by qRT-PCR (RIP qRT-PCR) using SYBR Green is performed to show that these transcripts are also highly upregulated in SCLC cell lines, H82 and H446.

To further interrogate the origins of these upregulated immunogenic nucleic acids, the genes and genome locations of PlaB induced A-RNA and Z-NA are elucidated by mapping results above to the human genome. Genes of origin for A-RNA overlap with Z-NA, and the genomic regions that harbor immunogenic Z-NAs are targeted.

To ensure reproducibility, J2 anti-dsRNA and Z22 anti-Z-NAs pulldown have n=2 biological replicates and qRT-PCR run in n=3 biological replicates using standard operating procedures. All statistical analysis, when applicable, are performed using one and two-way ANOVA, as well as Student T Test for comparison between conditions utilizing Prism v9. To ensure scientific rigor, all statistical tests with a p-value of <0.05 are considered significant. Regarding statistical analysis, when applicable, the data is presented as the average ±SEM. RNA-scq data analysis is performed by FCCC Bioinformatic Facility using best RNA-scq algorithm to thoroughly interrogate those enriched dsRNA and Z-NA species in treatment vs DMSO group.

Abundant A-RNA and Z-NA species are obtained after RIP to allow for multiple rounds of experiments. Given that A-RNA and Z-NA responses are specific to SF3B1 inhibition, direct aberrant splicing induced immunogenic species are elucidated after multiple rounds of filtering. Intron-retained mRNA, degraded or immature RNA, IncRNA, DNA/RNA hybrids, and other uncommon RNA species are specifically targeted. Furthermore, some accumulated endogenous nucleic acids in are expressed in SCLC cell lines. In addition, bulk J2- and Z22-enriched species are immuno-stimulatory and have unique structures that overlap and are derived from specific genome locations. Since there are no published data on aberrant splicing induced immuno-modulatory Z-NAs, the work described herein is innovative and allows us to further design A-RNA and Z-NA therapeutics and determine biomarkers to better treat aggressive SCLC. Due to deregulated transcriptome in cancer, SF3B1 inhibition induced Z-NAs may differ from MEFs. To confirm whether these induced Z-NAs are immuno-modulatory, individual Z-NA transcripts may be synthesized commercially or in vitro to better interrogate anti-tumor roles of these transcripts. However, observed anti-tumor response can be the result of the collective Z-NAs and not directly due to a single Z-NA species. In this instance, we will perform RIP-Seq with ZBP1 antibody that recognizes immunogenic Z-NAs, and intersect the results with Z22 antibody pulled down Z-NA to distinguish a subset of immunogenic Z-NA species.

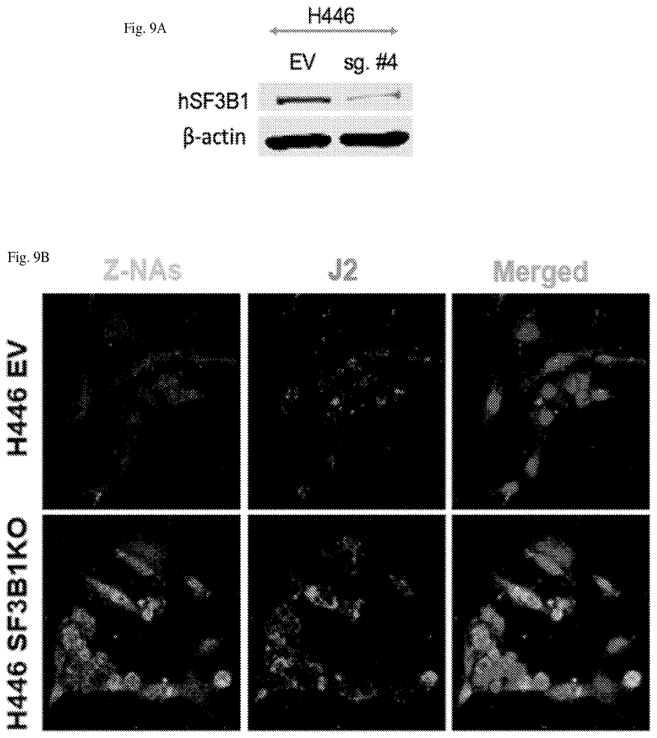

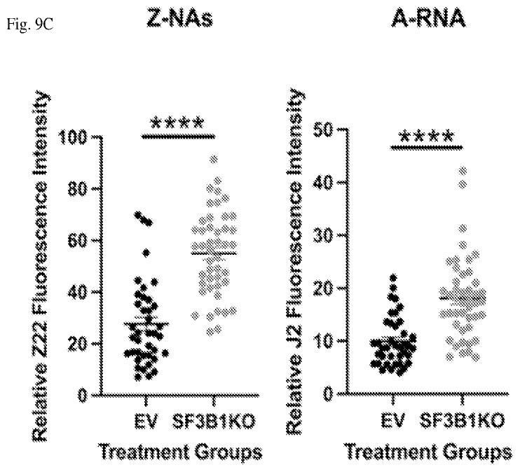

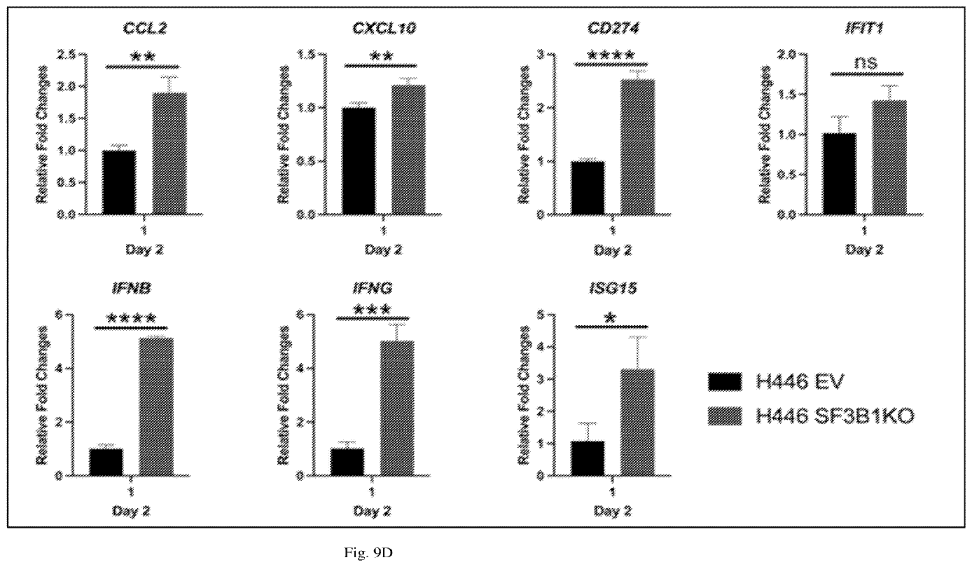

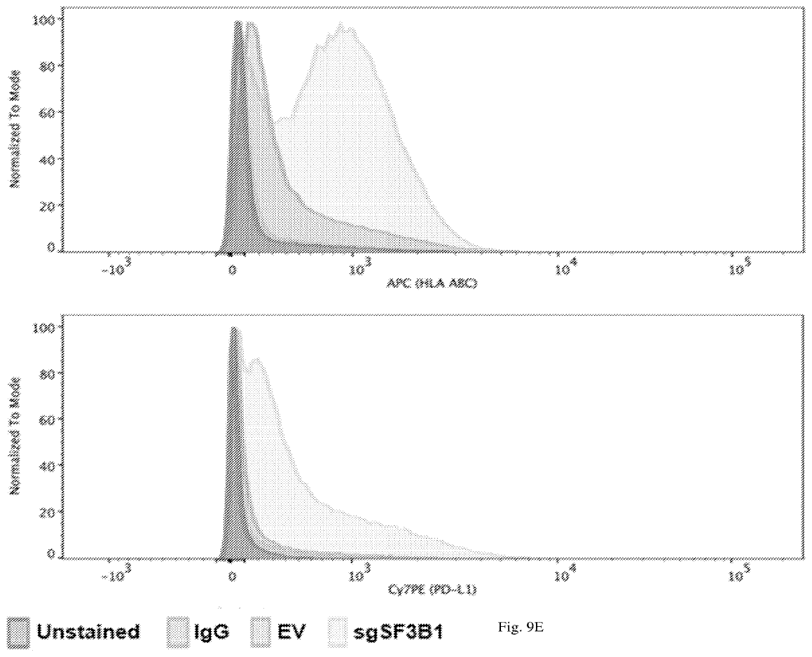

The effect of SF3B1 inhibition was further explored in H446 cells with SF3B1 knocked out. The SF3B1 knock out was confirmed using Western Blot Analysis. (FIG. 9A). Our data shows that SF3B1-KO cells exhibit enhanced Z-NA accumulation, thereby causing ZBP1-dependent necroptosis. (FIG. 9B). We then used immune-fluorescence (IF) technique to determine if both Z-NA and A-RNA were increased in the SF3B1-KO cells. We found that not only was there a robust induction of A-RNAs in the SF3B1-KO H446 cells, but a significant increase in left-handed nucleic acids (Z-NAs) (FIG. 9C).

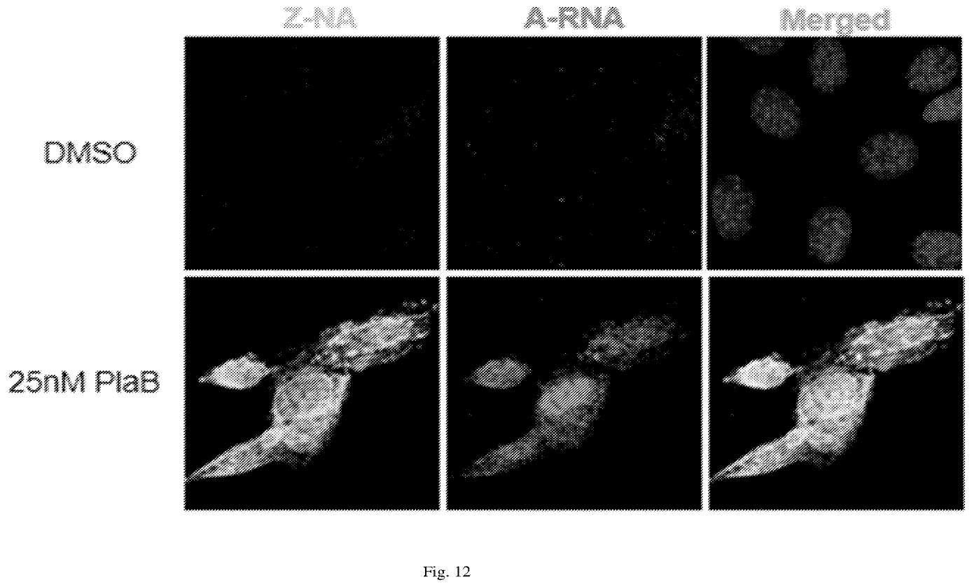

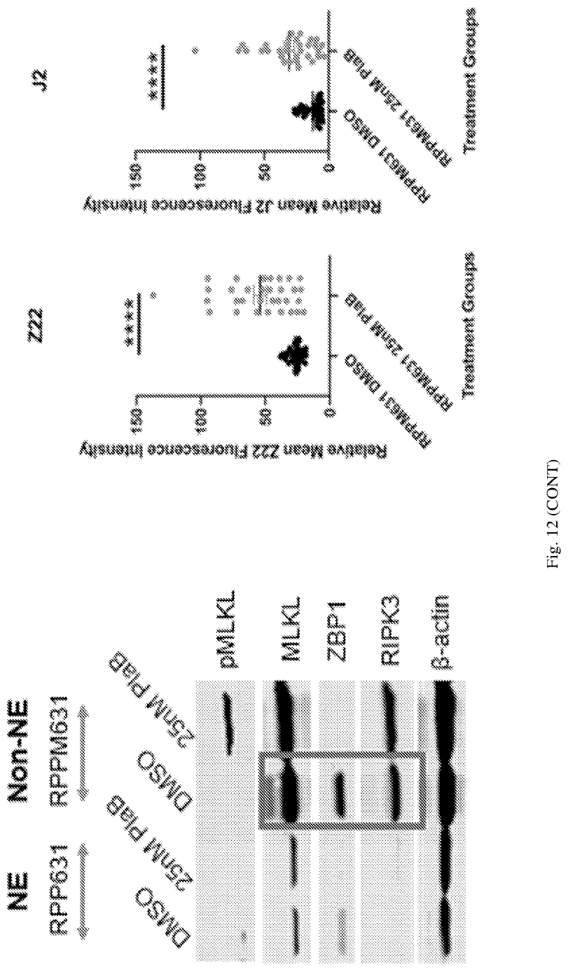

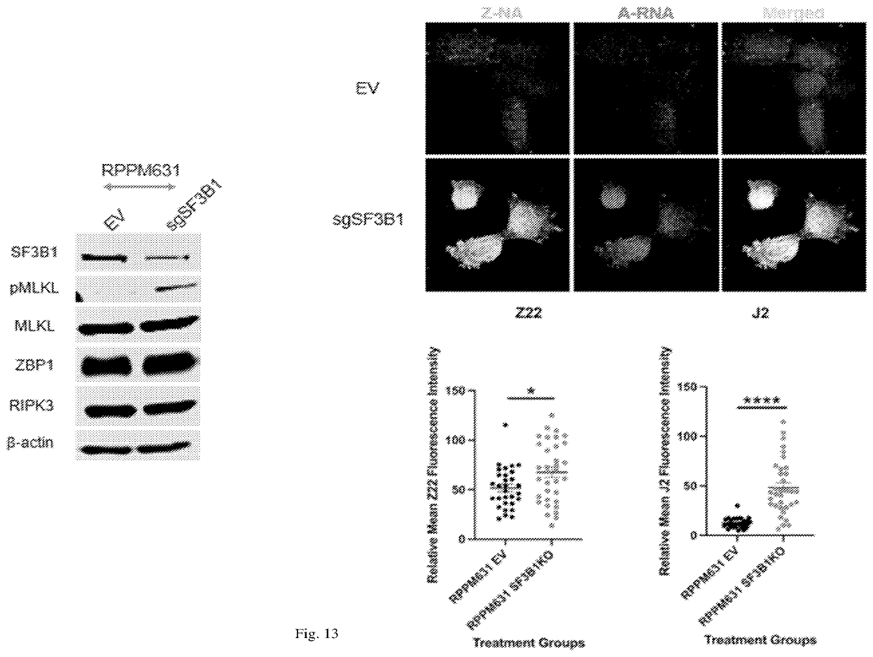

Additionally, SF3B1 inhibition was directly compared to SF3B1 knockout in the mouse SCLC cell line RPPM631. RPPM631 cells were treated with 25 nM PLAB and analyzed for their accumulation of Z-NA and A-RNA. PlaB treatment caused an increase in both Z-NA and A-RNA accumulation. (FIG. 12) Next, SF3B1-KO RPPM631 cells were generated and analyzed using immunc-fluorescence technique. The knock-out cells also showed an increase in the accumulation of Z-NA and A-RNA (FIG. 13).

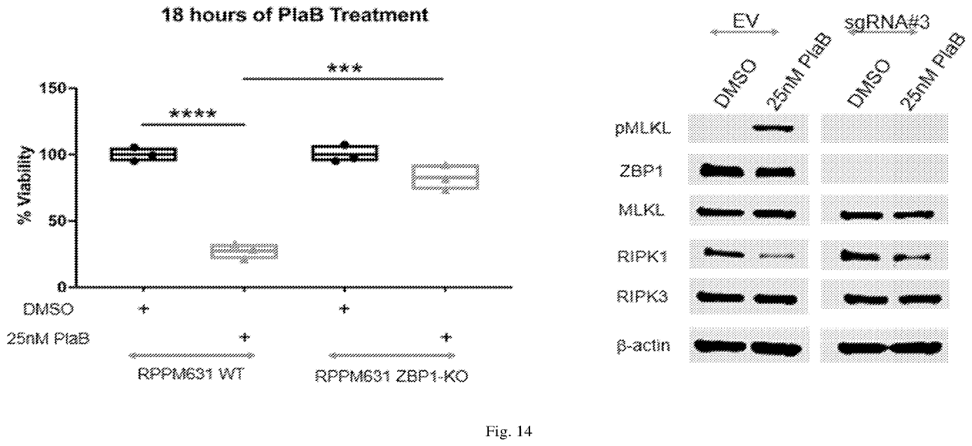

Next, we prepared ZBP1-KO RPPM631 cells to determine whether the necroptosis was ZBP-1 dependent. Wild type RPPM631 cells showed a significant decrease in viability when treated with PlaB but the ZBP1-KO RPPM631 cells showed only a minor decrease in viability. (FIG. 14) This indicates that the necroptosis is ZBP1 dependent.

Next, we determined whether the robust anti-tumor response and upregulation of IFN-β, IFN-γ, and IFN-responsive genes observed when SF3B1 is inhibited is also exhibited in SF3B1-KO cells. This was analyzed using rRT-qPCR in WT H446 cells and SF3B1-KO H446 cells. We observed that SF3B1 knock out triggered the same robust anti-tumor response and upregulation of IFN-β, IFN-γ, and IFN-responsive genes that was seen with SF3B1 inhibition. (FIG. 9D) We also confirmed that the robust IFN response in the SF3B1-KO H446 cells was sufficient to alter tumor-intrinsic immunogenicity. (FIG. 9E) We found that amplified IFN response strongly increased human leukocyte antigens ABC (HLA-ABC) and program death-ligand 1 (PD-L1) in H446 and H82 which indicates an effective ICB outcome.

### Example III: Effect of Spliceosome Inhibition on TIME Remodeling and Synergy with Anti-PD1 Immunotherapy

SCLC is an immunologically ‘COLD’ tumor characterized by an immuno-suppressive TME²¹⁻²³. Our data shows that SCLC are hyper-sensitive to spliceosome perturbation (FIG. 2). We observed an increase in IFN antiviral response (FIGS. 3A and 3B), HLA-ABC, and PD-L1 protein expression (FIGS. 3C and 3D). This is a phenotype that responds favorable to ICB therapies^{20,93-95}. Currently, whether tumor-intrinsic features can modulate immune-features in the TIME to facilitate ICB is unexplored. Despite promising advances in the use of immunotherapy, SCLC remains a devastating disease and only a very small fraction of SCLC patients respond to these therapies¹⁰⁻¹⁵. Also, the impact of spliceosome inhibitors on the TME and whether spliceosome inhibition can enhance immunotherapy response in SCLC remains completely unexplored. The hypothesis that spliceosome inhibition can increase tumor-intrinsic immunogenicity, as well as antigenicity, to reshape the TME and improve cancer immunotherapy is tested herein.

Our data shows that SF3B1 inhibition induces a favorable phenotype for immunotherapy, as one of the primary reasons for failed immunotherapy response is due to low antigenicity and the lack of infiltrating T cells^{20,96,97}. We hypothesize that spliceosome perturbation induced antiviral responses and tumor-features will re-activate immune surveillance. Assessment of the mechanism for spliceosome inhibition modulation of TIME interactions in immune-competent hosts uses SCLC mouse models derived cell lines: Rb1fl/flTrp53fl/flMycLSL/LSL (RPM) and Rb1fl/flTrp53fl/flPtenfl/fl (RPP) tumor models. 5×10⁶ tumor cells suspended in 50-100 μL of Matrigel (Corning) are injected subcutaneously into 4-5 weeks old C57BL/6J mice from the Jackson Laboratory. Tumors are treated intratumorally with 2.5 nM PlaB 2 weeks after tumor engraftment, and tumors are collected between 1500 and 2000 mm³ for downstream intracellular staining flow cytometry and IHC immune profiling. For flow cytometry analysis of tumor-associated immune, tumor chunks are homogenized into mononuclear cells using previously described methods⁹⁸. Then, cells then are fixed with 2% formaldehyde in PBS for 10 minutes at 37 °C, washed and permeabilized with ice-cold 90% methanol for 30 minutes, and washed for staining per manufacturer's instructions. For surface stains, staining is performed prior to permeabilization. The following immune cell populations are examined with the assistance of FCCC Cell Sorting Facility: B cells (CD19+), CD3+/CD4+ T cells, CD3+/CD8+ T cells, CD3+/Foxp3+ regulatory T cells, NK T cells (CD3+CD56+), monocytes (CD14+CD16+, CD14+CD16−, CD14−CD16+).

As for the IHC immune profiling, the tumor tissue is fixed in 10% formalin at 4 °C overnight, and subsequently dehydrated in 70% ethanol and embedded in paraffin, then sectioned at even intervals. Slides are deparaffinized and hydrated using xylene, graded ethyl alcohol, and dH₂O followed by antigen retrieval and stained with hematoxylin and eosin or appropriate immune cell specific antibodies. For IF staining of tumors, tumors are stained with PDGFRα, Z-NAs, and pMLKL following protocol described previously⁸⁰. The above-mentioned experiments allowed us to interrogate the impact of spliceosome inhibition on the TIME, and confirm whether necroptotic fibroblasts in the TIME potentiate anti-tumor response.

The status of the TIME is critical for tumor progression, immune surveillance, and response to immunotherapy⁹⁹. The effect of immune infiltrates recruited by spliceosome perturbation on further mitigation of tumor development alone or in combination with anti-PD-1 immunotherapy is also assessed. Here, 5×10⁶ tumor cells are engrafted subcutaneously into C57BL/6J mice described above. Once tumor reaches 150-250 mm³ it is intratumorally injected with 2.5 nM PlaB. Animals are co-treated with 200 μg of anti-PD-1 or IgG intrapcritoncally starting on day 7 after tumor challenge and twice a week thereafter for 6 weeks for a total of 12 doses. Tumor volumes is measured using calipers twice a week until 7-week timeframe is reached, Tumor volume across treatment conditions is plotted and analyzed using PRIMS v9.

To ensure reproducibility, 5 female mice and 5 male mice are tested per condition. The minimum number of animals required are used to accomplish these experiments to ensure scientific rigor based on G-power statistical analyses to achieve a power of 0.8, p-value 0.05, and anticipated effect size of 0.8. All statistical comparison between conditions are performed utilizing Prism v9. To ensure scientific rigor, all statistical tests with a p-value of <0.05 are considered significant. Regarding statistical analysis, when applicable, the data is presented as the average ±SEM.

Given a recent study of RBM39 splicing factor degradation in melanoma and colorectal cancers recruited T-cells and synergized with anti-PD-1 immunotherapy¹⁰⁰, SF3B1 inhibition will also recruit numerous immune populations, and synergize with anti-PD-1 immunotherapy. Alternatively, to reduce the number of animals used in these experiments, we can utilize novel cancer organoid models that recapitulates the TIME to directly test mouse tumor sensitivity to anti-PD-1 using ex vivo 3D microfluidic devices that are well described in the literature¹⁰¹⁻¹⁰³. Additionally, PlaB will drastically reduce tumor size, and may potentially eliminate all tumor growth based on in vitro results. Alternatively, RPP and RPM SCLC tumor models can be pre-treated for 72 hours in vitro with PlaB. Pre-treated cells are then inoculated into mice without further PlaB treatment. Additionally, H3B-8800, an enhanced analog of PlaB, can be used for in vivo experiments.

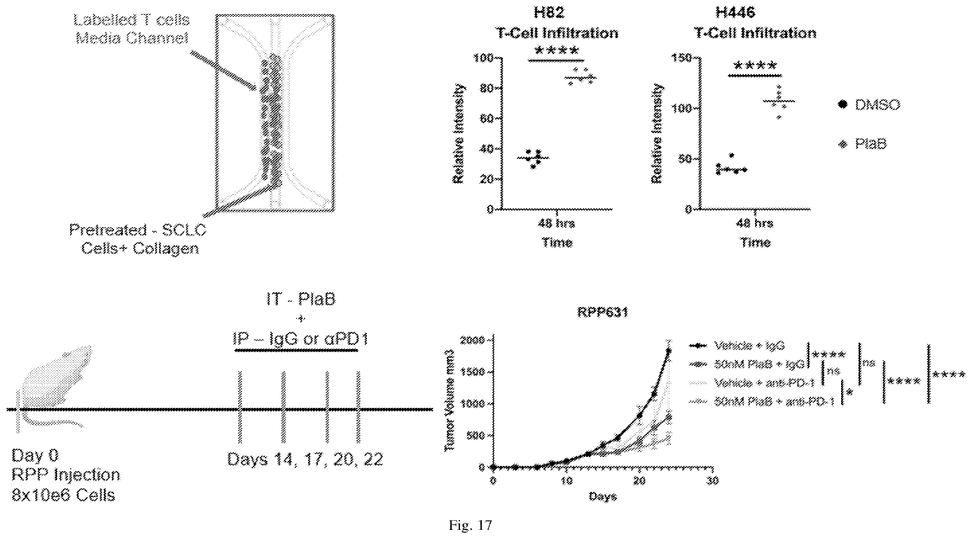

This combination therapy was tested in RPP631 cells in mice. At day 0, the mice were injected with 8×10⁶ RPP631 cells. At days 14, 17, 20, and 22, the mice were injected with PlaB or a vehicle intra-tumorally and IgG or αPD-1 intra-peritoneally. The tumors were then measured over 24 days. (FIG. 17). These results show that mice treated with PlaB and IgG or PlaB and anti-PD-1 had significantly reduced tumor size when compared to tumors treated with IgG or anti-PD1 only.

### Example IV: Spliceosome Inhibition Induces Cell Death in Necroptosis Incapable Tumors

Our data shows that spliceosome perturbation in SCLC drastically reduces cell survival (FIG. 2A), induces Z-NAs and apoptosis (FIGS. 4A and 4C), and RIPK1-KO rescues SCLC cell lines from PlaB induced cytotoxicity (FIG. 2B). Currently, ZBP1 and ADAR1 are the only two known proteins harbor Zα and interact with Z-NAs¹⁰⁴⁻¹⁰⁹. Our data shows an increase of ZBP1 (FIG. 4C) upon spliceosome perturbation, but the roles of ZBP1 and mechanism of cell death in SCLC are unclear due to downregulated RIPK3 and ZBP1 protein expression (data not shown). In addition, we have tested whether A-RNA response in tumor cells is responsible for antiviral innate immunity, however, knocking out the host mitochondrial antiviral signaling adaptor protein (MAVS) only minimally rescue the effects of PlaB-mediated cell death in H82 and H446 cell lines (data not shown).

Since RIPK1 mediated apoptosis can be triggered by TNF110, TNFR1 and TRADD-KO SCLC cell lines are generated herein for subsequent studies. Here, we utilize CRISPR-Cas9 KO to effectively knockout TNFR1 and TRADD in SCLC cells. To confirm depletion of TNFR1 and TRADD proteins, cell lysates are collected and western blot analysis is performed. The following experiments are performed using the TNFR1 and TRADD-KO SCLC cells generated above.

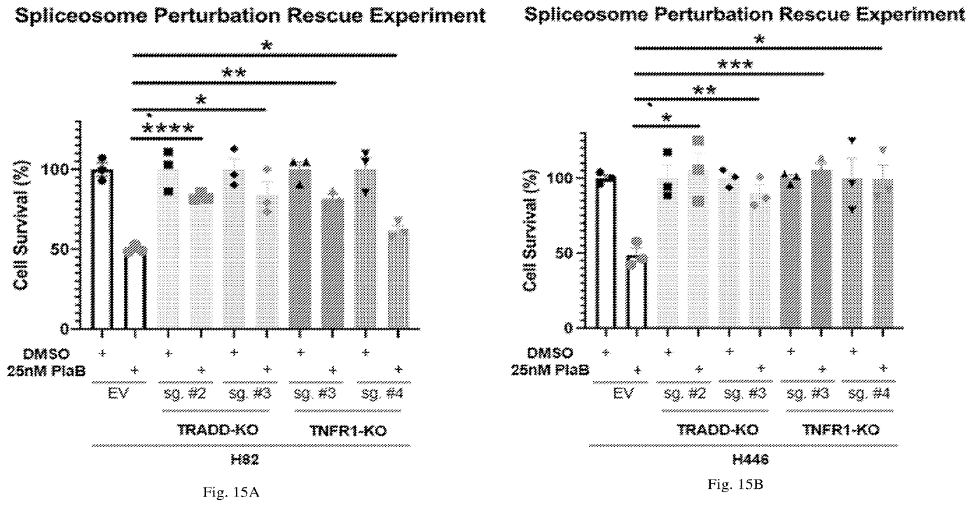

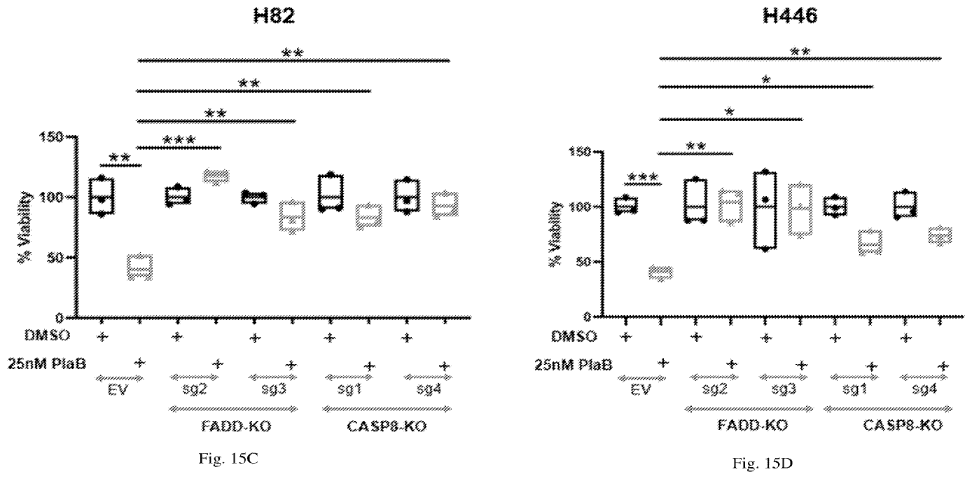

ZBP1 senses Z-NAs and has an important role in necroptosis⁷⁸⁻⁸⁴, since ZBP1 and RIPK3 are downregulated in SCLC cells, the hypothesis that knocking out TNF signaling mediated apoptosis will partially or fully rescue SCLC cell lines from PlaB induced cytotoxicity is tested. H82 and H446 WT and TNFR1 and TRADD-KO cells generated above are treated with DMSO or 2.5 nM PlaB for 18 hrs. Then, the cells are counted using 0.4% trypan blue solution assessing total viable cells and the percent of cell survived with the H82 and H446 WT cells is compared. (FIG. 15A-15B) This allows us to confirm whether TNF is partially or fully responsible for PlaB-mediated cell death in SCLC cell lines.

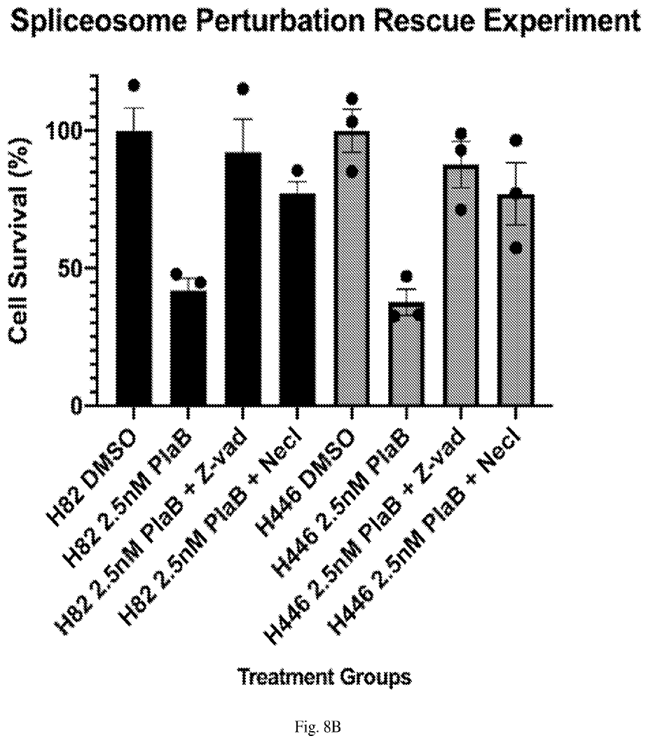

Our data shows that inhibiting the kinase activity of RIPK1 pharmacologically using necrostatin I (Nec-I) or inhibiting induction of caspase by Z-Vad FMK can significantly restore cell survival in H82 and H446 (FIG. 8). In addition, our CRISPR-Cas9 KO of RIPK1 also rescues SCLC cell lines from PlaB induced toxicity.

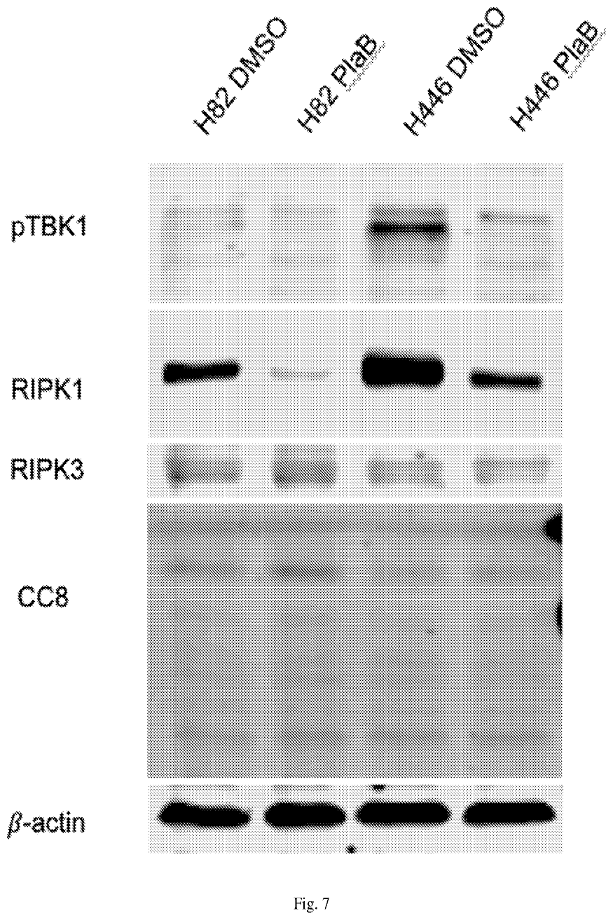

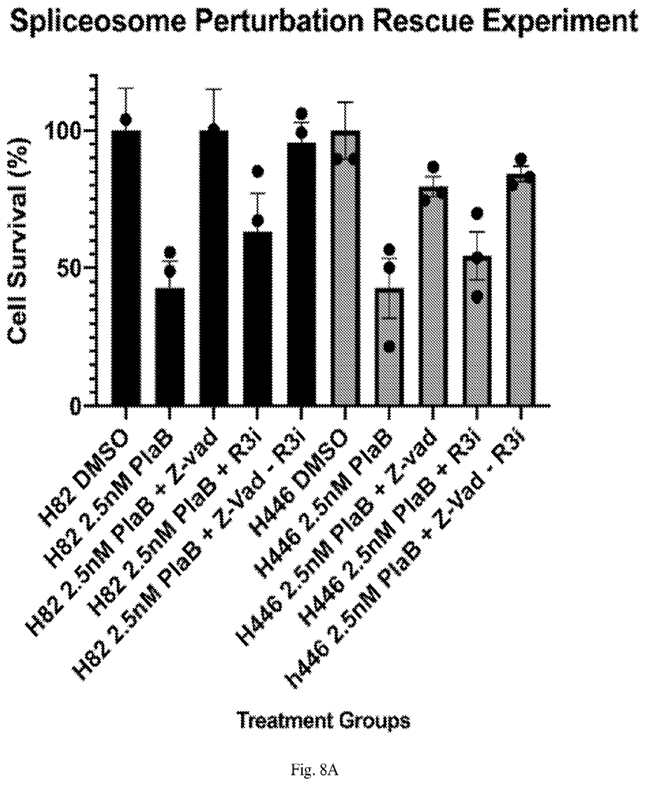

Some studies have reported that TBK1 suppresses TNF-induced cell death through pRIPK1¹¹¹⁻¹¹⁴, and our western blot analysis shows a dramatic reduction of pTBK1 protein (FIG. 7). Based on the publications and our findings, TNF sensing can at least partially rescue spliceosome perturbation mediated cell death. Alternatively, we can perform RNA-Seq and pathway analyses for WT vs RIPK1-KO cells treated with DMSO and PlaB to examine pathways that are downregulated and upregulated to precisely determine the pathways that have protective function against PlaB treatment.

Additionally, we analyzed the role played by FADD and CAPS8 in rescuing H82 and H446 cells from PlaB induced toxicity. Using the methods described above for the generation of TNFR1 and TRADD-KO cells, H82 and H446 FADD-KO cells and H82 and H446 CASP8-KO cells were generated. These cells were then treated with DMSO or 2.5 nM PlaB for 18 hrs and compared to WT H82 and H446 cells. As with TNFR1 and TRADD above, FADD or CASP8 effectively rescued the cells from PlaB toxicity. (FIG. 15C and FIG. 15D)

### Example V: Administration of Spliceosome Inhibitors Alone and in Combination Therapies in Human Patients

The information herein above can be applied clinically to patients for therapeutic intervention. A preferred embodiment of the invention comprises clinical application of the information described herein to a patient. This can occur after a patient arrives in the clinic and presents with cancer symptoms or after confirmation of a cancer diagnosis. The derived therapeutic dose of the spliceosome inhibitors disclosed herein for human would be in the ranges described above based on response rate and given at least once daily. One of ordinary skill in the art would be able to study the relevant factors and make the determination regarding the effective amount of the therapeutic compound without undue experimentation. In certain patients, the spliceosome inhibitor is administered via intertumoral administration. For this administration, the effective amount of the spliceosome inhibitor may be between 0-200 nM, 0-150 nM, 0-100 nM, 0-50 nM, 0-25 nM, 25 nm-200 nM, 25-150 nM, 25-100 nM, 25-50 nM, 50 nM-200 nM, 50-150 nM, 50-100 nM, 100-200 nM, 100-150 nM, 150 nM-200 nM, about 25 nM, about 50 nM, about 100 nM, about 150 nM or about 200 nM. In certain embodiments PlaB is administered via intratumural administration at anyone of the doses above.

In certain patients, the spliceosome inhibitor is administered via intraperitoneal administration. For this administration, the effective amount of the spliceosome inhibitor may be between 2-20 mg/kg, 0-20 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg. In certain embodiments PlaB is administered via intraperitoneal administration at anyone of the doses above.

In certain embodiments, the spliceosome inhibitor is administered via systemic or intravenous administration. In these embodiments, the effective amount of the spliceosome inhibitor may be between 2-10 mg/kg, 2-20 mg/kg, 0-20 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg.

In certain patients, PlaB is administered via systemic or intravenous administration at anyone of the doses above. In certain patients, PlaB is administered at a dose of 2-20 mg/kg.

In certain patients, sudemycin D6 is administered via systemic or intravenous administration at anyone of the doses above. In certain patients, sudemycin D6 is administered at a dose of 14 mg/kg-50 mg/kg.

In certain patients, indisulam is administered via systemic or intravenous administration at anyone of the doses above. In certain patients, indisulam is administered at a dose of 12.5 mg/kg-100 mg/kg.

In certain embodiments, the spliceosome inhibitor is administered via oral administration. In these embodiments, the effective amount of the spliceosome inhibitor may be between 2-10 mg/kg, 2-20 mg/kg, 0-20 mg/kg, 0-40 mg/kg, 2-40 mg/kg, 10-40 mg/kg, 10-20 mg/kg, 20-40 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, or about 40 mg/kg. In certain embodiments H3B-8800 is administered via oral administration at anyone of the doses above.

For any method of administration, the spliceosome modulator may be selected from pladienolide D, pladienolide B, pladienolide A, pladienolide C, pladienolide E, pladienolide F, pladienolide G, H3B-8800, indisulam, herboxidiene, spliceostatin, sudemycin, FR901463, FR901464, FR901465, splicostatins A-G, thailanstatins A-C, meamycins, E7107, FD-895, herboxidiene, GEX1A, GEX1Q1-5, RQN-18690A (18-deoxyherboxidiene), sudemycins C1, sudemycins D1, sudemycins F1, sudemycins E, sudemycins D6, isoginkgetin, madrasin, tetrocarcin A, N-palmitoyl-L-leucine, psoromic acid, clotrimazole, NSC635326, napthazarin and/or derivatives thereof. The inhibitors described herein have been shown to be well tolerated and the symptoms were assessed using clinical scores criteria.

The treatment protocol can also optionally include administration of effective amounts of one or more of additional therapeutic agents that treat or inhibit cancer. The treatment protocol can also optionally include co-administration of additional chemotherapeutics. In certain patients, treatment using two or more substances improves the therapy when compared to using either one of the substances alone, by maximizing efficacy, reducing toxicity, and addressing interpatient variability, as well as delaying and/or overcoming innate or acquired resistance.

Additionally, the combination therapies may be effective to reduce the effective amount of at least one of the spliceosome inhibitor and the second therapy. In certain embodiments, the effective amount of the spliceosome inhibitor and/or second therapy is reduced by 75%, 85%, 90%, 95%, or greater when compared to solo treatment. The effective amount can be reduced by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

### REFERENCES

1. Siegel R L, Miller K D, Fuchs H E, Jemal A. Cancer statistics, 2022. CA Cancer J Clin. 2022 Jan;72(1): 7-33. doi: 10.3322/caac.21708. Epub 2022 Jan. 12. PMID: 35020204.
2. Campbell A M, Campling B G, Algazy K M, el-Deiry W S. Clinical and molecular features of small cell lung cancer. Cancer Biol Ther. 2002 Mar-Apr;1(2):105-12. doi: 10.4161/cbt.52. PMID: 12170769.
3. Siegel R L, Miller K D, Jemal A. Cancer statistics, 2016. CA Cancer J Clin. 2016 Jan-Feb;66(1):7-30. doi:10.3322/caac.21332. Epub 2016 Jan. 7. PMID: 26742998.
4. Howlader N, Noone A M, Krapcho M, Miller D, Brest A, Yu M, Ruhl J, Tatalovich Z, Mariotto A, Lewis D R, Chen H S, Feuer E J, Cronin K A (eds). SEER Cancer Statistics Review, 1975-2016, National Cancer Institute. Bethesda, MD. https://seer.cancer.gov/csr/1975_2016/, based on November 2018 SEER data submission, posted to the SEER web site, April 2019.
5. Hann C, Wu M A, Rekhtman N, Rudin C M. Cancer: Principles and Practice of Oncology. 11th ed. Philadelphia (USA): Wolters Kluwer, c2019. Chapter 49, Small Cell and Neuroendocrine Tumors of the Lung, p671-99.
6. Bernhardt E B, Jalal S I. Small Cell Lung Cancer. Cancer Treat Res. 2016;170:301-22. doi: 10.1007/978-3-319-40389-2_14. PMID: 27535400.
7. Wang S, Tang J, Sun T, Zheng X, Li J, Sun H, Zhou X, Zhou C, Zhang H, Cheng Z, Ma H, Sun H. Survival changes in patients with small cell lung cancer and disparities between different sexes, socioeconomic statuses and ages. Sci Rep. 2017 May 2;7(1):1339. doi: 10.1038/s41598-017-01571-0. PMID: 28465554; PMCID: PMC5431017.
8. Byers L A, Rudin C M. Small cell lung cancer: where do we go from here? Cancer. 2015 Mar. 1;121(5):664-72. doi: 10.1002/cncr.29098. Epub 2014 Oct. 21. PMID: 25336398; PMCID: PMC5497465.
9. Cuevas L M, Daud A I. Immunotherapy for melanoma. Semin Cutan Med Surg. 2018 Jun;37(2): 127-131. doi: 10.12788/j.sder.2018.028. PMID: 30040090.
10. Horn L, Mansfield A S, Szczęsna A, Havel L, Krzakowski M, Hochmair M J, Huemer F, Losonczy G, Johnson M L, Nishio M, Reck M, Mok T, Lam S, Shames D S, Liu J, Ding B, Lopez-Chavez A, Kabbinavar F, Lin W. Sandler A, Liu s V; IMpower133 Study Group. First-Line Atezolizumab plus Chemotherapy in Extensive-Stage Small-Cell Lung Cancer. N Engl J Med. 2018 Dec. 6;379(23):2220-2229. doi: 10.1056/NEJMoa1809064. Epub 2018 Sep. 25. PMID: 30280641.
11. Paz-Ares L, Dvorkin M, Chen Y, Reinmuth N, Hotta K, Trukhin D, Statsenko G, Hochmair M J, Özgüroǧlu M, Ji J H. Voitko O, Poltoratskiy A, Ponce S, Verderame F, Havel L, Bondarenko I, Kazarnowicz A. Losonczy G, Conev N V, Armstrong J, Byrne N, Shire N, Jiang H, Goldman J W; CASPIAN investigators. Durvalumab plus platinum-etoposide versus platinum-etoposide in first-line treatment of extensive-stage small-cell lung cancer (CASPIAN): a randomised, controlled, open-label, phase 3 trial. Lancet. 2019 Nov. 23;394(10212):1929-1939. doi: 10.1016/S0140-6736(19)32222-6. Epub 2019 Oct. 4. PMID: 31590988.
12. Goldman J W, Dvorkin M, Chen Y, Reinmuth N, Hotta K, Trukhin D, Statsenko G, Hochmair M J, Özgüroǧlu M, Ji J H, Garassino M C, Voitko O, Poltoratskiy A, Ponce S, Verderame F, Havel L, Bondarenko I, Każarnowicz A, Losonczy G, Conev N V, Armstrong J, Byrne N, Thiyagarajah P, Jiang H, Paz-Ares L; CASPIAN investigators. Durvalumab, with or without tremelimumab, plus platinum-etoposide versus platinum-etoposide alone in first-line treatment of extensive-stage small-cell lung cancer (CASPIAN): updated results from a randomised, controlled, open-label, phase 3 trial. Lancet Oncol. 2021 Jan;22(1):51-65. doi: 10.1016/S1470-2045(20)30539-8. Epub 2020 Dec. 4. PMID: 33285097.
13. Rudin C M, Awad M M, Navarro A, Gottfried M, Peters S. Csőszi T, Cheema P K, Rodriguez-Abreu D, Wollner M, Yang J C, Mazieres J, Orlandi F J, Luft A, Gümüş M, Kato T, Kalemkerian G P, Luo Y, Ebiana V, Pietanza M C, Kim H R; KEYNOTE-604 Investigators. Pembrolizumab or Placebo Plus Etoposide and Platinum as First-Line Therapy for Extensive-Stage Small-Cell Lung Cancer: Randomized, Double-Blind, Phase III KEYNOTE-604 Study. J Clin Oncol. 2020 Jul. 20;38(21):2369-2379. doi: 10.1200/JCO.20.00793. Epub 2020 May 29. PMID: 32468956; PMCID: PMC7474472.
14. Chung H C, Piha-Paul S A, Lopez-Martin J, Schellens J H M, Kao S, Miller W H Jr, Delord J P, Gao B, Planchard D, Gottfried M. Zer A, Jalal S I, Penel N, Mehnert J M, Matos I, Bennouna J, Kim D W, Xu L, Krishnan S. Norwood K, Ott P A. Pembrolizumab After Two or More Lines of Previous Therapy in Patients With Recurrent or Metastatic SCLC: Results From the KEYNOTE-028 and KEYNOTE-158 Studies. J Thorac Oncol. 2020 Apr;15(4):618-627. doi: 10.1016/j.jtho.2019.12.109. Epub 2019 Dec. 20. PMID: 31870883.
15. Spigel D R, Vicente D, Ciuleanu T E, Gettinger S, Peters S, Horn L, Audigier-Valette C, Pardo Aranda N, Juan-Vidal O, Cheng Y, Zhang H, Shi M, Luft A, Wolf J, Antonia S, Nakagawa K, Fairchild J, Baudelet C, Pandya D, Doshi P, Chang H, Reck M. Second-line nivolumab in relapsed small-cell lung cancer: CheckMate 331☆. Ann Oncol. 2021 May;32(5):631-641. doi: 10.1016/j.annonc.2021.01.071. Epub 2021 Feb. 1. PMID: 33539946.
16. Farago A F, Keane F K. Current standards for clinical management of small cell lung cancer. Transl Lung Cancer Res. 2018 Feb;7(1):69-79. doi: 10.21037/tler.2018.01.16. PMID: 29535913; PMCID: PMC5835595.
17. Peifer M, Fernández-Cuesta L, Sos M L, George J, Seidel D, Kasper L H, Plenker D, Leenders F, Sun R, Zander T, Menon R. Koker M. Dahmen I, Müller C, Di Cerbo V, Schildhaus H U, Altmüller J, Baessmann I, Becker C, de Wilde B, Vandesompele J, Böhm D, Ansén S, Gabler F, Wilkening I, Heynck S, Heuckmann J M, Lu X, Carter S L, Cibulskis K, Banerji S, Getz G, Park K S, Rauh D, Grütter C, Fischer M, Pasqualucci L, Wright G, Wainer Z, Russell P, Petersen I, Chen Y, Stoelben E, Ludwig C, Schnabel P. Hoffmann H, Muley T. Brockmann M. Engel-Riedel W, Muscarella L A, Fazio V M, Groen H, Timens W, Sietsma H, Thunnissen E, Smit E, Heideman D A, Snijders P J, Cappuzzo F, Ligorio C, Damiani S, Field J, Solberg S, Brustugun O T, Lund-Iversen M, Sänger J, Clement J H, Soltermann A, Moch H, Weder W, Solomon B, Soria J C, Validire P, Besse B, Brambilla E, Brambilla C, Lantuejoul S, Lorimier P, Schneider P M, Hallek M, Pao W, Meyerson M, Sage J, Shendure J, Schneider R. Büttner R, Wolf J, Nürnberg P, Perner S, Heukamp L C, Brindle P K, Haas S, Thomas R K. Integrative genome analyses identify key somatic driver mutations of small-cell lung cancer. Nat Genet. 2012 Oct;44(10):1104-10. doi: 10.1038/ng.2396. Epub 2012 Sep. 2. PMID: 22941188; PMCID: PMC4915822.
18. George J, Lim J S, Jang S J, Cun Y, Ozretic L, Kong G, Leenders F, Lu X, Fernández-Cuesta L, Bosco G, Müller C, Dahmen I, Jahchan N S, Park K S, Yang D, Karnezis A N, Vaka D. Torres A, Wang M S, Korbel J O, Menon R, Chun S M, Kim D, Wilkerson M, Hayes N, Engelmann D, Pützer B, Bos M, Michels S, Vlasic I, Seidel D, Pinther B, Schaub P, Becker C, Altmüller J, Yokota J, Kohno T, Iwakawa R, Tsuta K, Noguchi M, Muley T, Hoffmann H, Schnabel P A, Petersen I, Chen Y, Soltermann A, Tischler V. Choi C M, Kim Y H, Massion P P, Zou Y, Jovanovic D, Kontic M, Wright G M, Russell P A, Solomon B, Koch I, Lindner M, Muscarella L A, la Torre A, Field J K, Jakopovic M, Knezevic J, Castaños-Vélez E, Roz L, Pastorino U, Brustugun O T, Lund-Iversen M, Thunnissen E, Köhler J, Schuler M, Botling J, Sandelin M, Sanchez-Cespedes M, Salvesen H B, Achter V, Lang U, Bogus M, Schneider P M, Zander T, Ansen S, Hallek M, Wolf J, Vingron M, Yatabe Y, Travis W D, Nürnberg P, Reinhardt C, Perner S, Heukamp L, Büttner R, Haas S A, Brambilla E, Peifer M, Sage J, Thomas R K. Comprehensive genomic profiles of small cell lung cancer. Nature. 2015 Aug. 6;524(7563):47-53. doi: 10.1038/nature14664. Epub 2015 Jul. 13. PMID: 26168399; PMCID: PMC4861069.
19. Rudin C M, Brambilla E, Faivre-Finn C, Sage J. Small-cell lung cancer. Nat Rev Dis Primers. 2021 Jan. 14;7(1):3. doi: 10.1038/s41572-020-00235-0. PMID: 33446664; PMCID: PMC8177722.
20. Hegde P S, Chen D S. Top 10 Challenges in Cancer Immunotherapy. Immunity. 2020 Jan 14;52(1):17-35. doi: 10.1016/j.immuni.2019.12.011. PMID: 31940268.
21. Doyle A, Martin W J, Funa K, Gazdar A, Carney D, Martin S E, Linnoila I, Cuttitta F, Mulshine J, Bunn P, et al. Markedly decreased expression of class I histocompatibility antigens, protein, and mRNA in human small-cell lung cancer. J Exp Med. 1985 May 1;161(5):1135-51. doi: 10.1084/jem.161.5.1135.PMID: 2580935; PMCID: PMC2187608.
22. Funa K, Gazdar A F, Minna J D, Linnoila R I. Paucity of beta 2-microglobulin expression on small cell lung cancer, bronchial carcinoids and certain other neuroendocrine tumors. Lab Invest. 1986 Aug;55(2): 186-93. PMID: 3525983.
23. Restifo N P, Esquivel F, Kawakami Y, Yewdell J W, Mulé J J, Rosenberg S A, Bennink J R. Identification of human cancers deficient in antigen processing. J Exp Med. 1993 Feb. 1;177(2):265-72. doi: 10.1084/jem.177.2.265. PMID: 8426105; PMCID: PMC1950463.
24. Schumacher T N, Schreiber R D. Neoantigens in cancer immunotherapy. Science. 2015 Apr. 3;348(6230):69-74. doi: 10.1126/science.aaa4971. PMID: 25838375.
25. Mahadevan N R, Knelson E H, Wolff J O, Vajdi A, Saigí M, Campisi M, Hong D, Thai T C, Piel B, Han S, Reinhold B B, Duke-Cohan J S, Poitras M J, Taus L J, Lizotte P H, Portell A, Quadros V, Santucci A D, Murayama T, Cañadas I, Kitajima S, Akitsu A, Fridrikh M, Watanabe H, Reardon B, Gokhale P C, Paweletz C P, Awad M M, Van Allen E M, Lako A, Wang X T, Chen B, Hong F, Sholl L M, Tolstorukov M Y, Pfaff K, Janne P A, Gjini E, Edwards R, Rodig S, Reinherz E L, Oser M G, Barbie D A. Intrinsic Immunogenicity of Small Cell Lung Carcinoma Revealed by Its Cellular Plasticity. Cancer Discov. 2021 Aug;11(8):1952-1969. doi: 10.1158/2159-8290.CD-20-0913. Epub 2021 Mar. 11. PMID: 33707236; PMCID: PMC8338750.
26. Pan Q, Shai O, Lee L J, Frey B J, Blencowe B J. Deep surveying of alternative splicing complexity in the human transcriptome by high-throughput sequencing. Nat Genet. 2008 Dec;40(12):1413-5. doi:10.1038/ng.259. Epub 2008 Nov. 2. Erratum in: Nat Genet. 2009 Jun;41(6):762. PMID: 18978789.
27. Chen W, Moore M J. Spliceosomes. Curr Biol. 2015 Mar. 2;25(5):R181-3. doi: 10.1016/j.cub.2014.11.059. PMID: 25734262.
28. Wilkinson M E, Charenton C, Nagai K. RNA Splicing by the Spliceosome. Annu Rev Biochem. 2020 Jun. 20;89:359-388. doi:10.1146/annurev-biochem-091719-064225. Epub 2019 Dec. 3. PMID: 31794245.
29. Hegele A, Kamburov A, Grossmann A, Sourlis C, Wowro S, Weimann M, Will C L, Pena V, Lührmann R, Stelzl U. Dynamic protein-protein interaction wiring of the human spliceosome. Mol Cell. 2012 Feb. 24:45(4):567-80. doi: 10.1016/j.molcel.2011.12.034. PMID: 22365833.
30. Seiler M, Peng S, Agrawal A A, Palacino J, Teng T, Zhu P, Smith P G; Cancer Genome Atlas Research Network, Buonamici S, Yu L. Somatic Mutational Landscape of Splicing Factor Genes and Their Functional Consequences across 33 Cancer Types. Cell Rep. 2018 Apr. 3;23(1):282-296.e4. doi: 10.1016/j.celrep.2018.01.088. PMID: 29617667: PMCID: PMC5933844.
31. Martelotto L G, De Filippo M R, Ng C K, Natrajan R, Fuhrmann L, Cyrta J, Piscuoglio S, Wen H C, Lim R S, Shen R, Schultheis A M, Wen Y H, Edelweiss M, Mariani O, Stenman G, Chan T A, Colombo P E, Norton L, Vincent-Salomon A, Reis-Filho J S, Weigelt B. Genomic landscape of adenoid cystic carcinoma of the breast. J Pathol. 2015 Oct;237(2):179-89. doi: 10.1002/path.4573. Epub 2015 Jul. 14. PMID: 26095796; PMCID: PMC4676955.
32. Seiler M, Yoshimi A, Darman R, Chan B, Keaney G. Thomas M, Agrawal A A, Caleb B, Csibi A, Sean E, Fekkes P, Karr C, Klimek V, Lai G, Lee L, Kumar P, Lee S C, Liu X, Mackenzie C, Meeske C, Mizui Y, Padron E, Park E, Pazolli E, Peng S, Prajapati S, Taylor J, Teng T, Wang J, Warmuth M, Yao H, Yu L, Zhu P, Abdel-Wahab O, Smith P G, Buonamici S. H3B-8800, an orally available small-molecule splicing modulator, induces lethality in spliceosome-mutant cancers. Nat Med. 2018 May;24(4):497-504. doi: 10.1038/nm.4493. Epub 2018 Feb. 19. PMID: 29457796; PMCID: PMC6730556.
33. Lee S C, Abdel-Wahab O. Therapeutic targeting of splicing in cancer. Nat Med. 2016 Sep. 7;22(9):976-86. doi: 10.1038/nm.4165. PMID: 27603132; PMCID: PMC5644489.
34. Hsu T Y, Simon L M, Neill N J, Marcotte R, Sayad A, Bland C S, Echeverria G V, Sun T, Kurley S J, Tyagi S, Karlin K L, Dominguez-Vidaña R, Hartman J D, Renwick A, Scorsone K, Bernardi R J, Skinner S O, Jain A, Orellana M, Lagisetti C, Golding I, Jung S Y, Neilson J R, Zhang X H, Cooper T A, Webb T R, Neel B G, Shaw C A, Westbrook T F. The spliceosome is a therapeutic vulnerability in MYC-driven cancer. Nature. 2015 Sep. 17;525(7569):384-8. doi: 10.1038/nature14985. Epub 2015 Sep. 2. PMID: 26331541; PMCID: PMC4831063.
35. Koh C M, Bezzi M, Low D H, Ang W X, Teo S X, Gay F P, Al-Haddawi M, Tan S Y, Osato M, Sabò A, Amati B, Wee K B, Guccione E. MYC regulates the core pre-mRNA splicing machinery as an essential step in lymphomagenesis. Nature. 2015 Jul. 2;523(7558):96-100. doi: 10.1038/nature14351. Epub 2015 May 11. PMID: 25970242.
36. Seiler M, Yoshimi A, Darman R, Chan B, Keaney G, Thomas M, Agrawal A A, Caleb B, Csibi A, Sean E, Fekkes P, Karr C, Klimek V, Lai G, Lee L, Kumar P, Lee S C, Liu X, Mackenzie C, Meeske C, Mizui Y, Padron E, Park E, Pazolli E, Peng S, Prajapati S, Taylor J. Teng T, Wang J, Warmuth M. Yao H, Yu L, Zhu P, Abdel-Wahab O, Smith P G, Buonamici S. H3B-8800, an orally available small-molecule splicing modulator, induces lethality in spliceosome-mutant cancers. Nat Med. 2018 May;24(4):497-504. doi: 10.1038/nm.4493. Epub 2018 Feb. 19. PMID: 29457796; PMCID: PMC6730556.
37. Bowling E A, Wang J H, Gong F, Wu W, Neill N J, Kim I S, Tyagi S, Orellana M, Kurley S J, Dominguez-Vidaña R, Chung H C, Hsu T Y, Dubrulle J, Saltzman A B, Li H, Meena J K, Canlas G M, Chamakuri S, Singh S, Simon L M, Olson C M, Dobrolecki L E, Lewis M T, Zhang B, Golding I, Rosen J M, Young D W, Malovannaya A, Stossi F, Miles G, Ellis M J, Yu L, Buonamici S, Lin C Y, Karlin K L, Zhang X H, Westbrook T F. Spliceosome-targeted therapies trigger an antiviral immune response in triple-negative breast cancer. Cell. 2021 Jan. 21;184(2):384-403.e21. doi: 10.1016/j.cell.2020.12.031. Epub 2021 Jan. 14. PMID: 33450205; PMCID: PMC8635244.
38. Sun C. The SF3b complex: splicing and beyond. Cell Mol Life Sci. 2020 Sep; 77(18):3583-3595. doi: 10.1007/s00018-020-03493-z. Epub 2020 Mar. 5. PMID: 32140746; PMCID: PMC7452928.
39. Biankin A V, Waddell N, Kassahn K S, Gingras M C, Muthuswamy L B, Johns A L, Miller D K, Wilson P J, Patch A M, Wu J, Chang D K, Cowley M J, Gardiner B B, Song S, Harliwong I, Idrisoglu S, Nourse C, Nourbakhsh E, Manning S, Wani S, Gongora M, Pajic M, Scarlett C J, Gill A J, Pinho A V, Rooman I, Anderson M, Holmes O, Leonard C. Taylor D, Wood S, Xu Q, Nones K, Fink J L, Christ A, Bruxner T, Cloonan N, Kolle G, Newell F, Pinese M, Mead R S, Humphris J L, Kaplan W, Jones M D, Colvin E K, Nagrial A M, Humphrey E S, Chou A, Chin V T, Chantrill L A, Mawson A, Samra J S, Kench J G, Lovell J A, Daly R J, Merrett N D, Toon C, Epari K, Nguyen N Q, Barbour A. Zeps N: Australian Pancreatic Cancer Genome Initiative, Kakkar N, Zhao F, Wu Y Q, Wang M, Muzny D M, Fisher W E, Brunicardi F C, Hodges S E, Reid J G, Drummond J, Chang K, Han Y, Lewis L R, Dinh H, Buhay C J, Beck T, Timms L, Sam M, Begley K, Brown A, Pai D, Panchal A, Buchner N, De Borja R, Denroche R E, Yung C K, Serra S. Onetto N. Mukhopadhyay D, Tsao M S, Shaw P A, Petersen G M, Gallinger S, Hruban R H, Maitra A, Iacobuzio-Donahue C A, Schulick R D, Wolfgang C L, Morgan R A, Lawlor R T, Capelli P, Corbo V, Scardoni M, Tortora G, Tempero M A, Mann K M, Jenkins N A, Perez-Mancera P A, Adams D J, Largaespada D A, Wessels L F, Rust A G, Stein L D, Tuveson D A, Copeland N G, Musgrove E A, Scarpa A, Eshleman J R. Hudson T J, Sutherland R L, Wheeler D A, Pearson J V, McPherson J D, Gibbs R A, Grimmond S M. Pancreatic cancer genomes reveal aberrations in axon guidance pathway genes. Nature. 2012 Nov. 15;491(7424):399-405. doi: 10.1038/nature11547. Epub 2012 Oct. 24. PMID: 23103869; PMCID: PMC3530898.
40. Cancer Genome Atlas Network. Genomic Classification of Cutaneous Melanoma. Cell. 2015 Jun. 18;161(7):1681-96. doi: 10.1016/j.cell.2015.05.044. PMID: 26091043; PMCID: PMC4580370.
41. Hintzsche J D, Gorden N T, Amato C M, Kim J, Wuensch K E, Robinson S E, Applegate A J, Couts K L, Medina T M, Wells K R, Wisell J A, McCarter M D, Box N F, Shellman Y G, Gonzalez R C, Lewis K D, Tentler J J, Tan A C, Robinson W A. Whole-exome sequencing identifies recurrent SF3B1 R625 mutation and comutation of NF1 and KIT in mucosal melanoma. Melanoma Res. 2017 Jun;27(3):189-199. doi: 10.1097/CMR.0000000000000345. PMID: 28296713: PMCID: PMC5470740.
42. Martin M, Maßhöfer L, Temming P, Rahmann S, Metz C, Bornfeld N, van de Nes J, Klein-Hitpass L, Hinnebusch A G, Horsthemke B, Lohmann D R, Zeschnigk M. Exome sequencing identifies recurrent somatic mutations in EIF1AX and SF3B1 in uveal melanoma with disomy 3. Nat Genet. 2013 Aug;45(8):933-6. doi: 10.1038/ng.2674. Epub 2013 Jun. 23. PMID: 23793026; PMCID: PMC4307600.
43. Quesada V, Conde L, Villamor N. Ordóñez G R, Jares P, Bassaganyas L, Ramsay A J, Beà S, Pinyol M, Martínez-Trillos A, López-Guerra M, Colomer D, Navarro A, Baumann T, Aymerich M, Rozman M, Delgado J, Giné E, Hernández J M, González-Díaz M, Puente D A, Velasco G, Freije J M, Tubío J M, Royo R, Gelpí J L, Orozco M, Pisano D G, Zamora J, Vázquez M, Valencia A, Himmelbauer H, Bayes M, Heath S, Gut M, Gut I, Estivill X, López-Guillermo A, Puente X S, Campo E, López-Otín C. Exome sequencing identifies recurrent mutations of the splicing factor SF3B1 gene in chronic lymphocytic leukemia. Nat Genet. 2011 Dec. 11;44(1):47-52. doi: 10.1038/ng.1032. PMID: 22158541.
44. Cancer Genome Atlas Network. Comprehensive molecular portraits of human breast tumours. Nature. 2012 Oct. 4;490(7418):61-70. doi: 10.1038/nature11412. Epub 2012 Sep. 23. PMID: 23000897; PMCID: PMC3465532.
45. Jung H, Lee D, Lee J, Park D, Kim Y J, Park W Y, Hong D, Park P J, Lee E. Intron retention is a widespread mechanism of tumor-suppressor inactivation. Nat Genet. 2015 Nov; 47(11):1242-8. doi: 10.1038/ng.3414. Epub 2015 Oct. 5. PMID: 26437032.
46. Zheng J T, Lin C X, Fang Z Y, Li H D. Intron Retention as a Mode for RNA-Seq Data Analysis. Front Genet. 2020 Jul 7;11:586. doi: 10.3389/fgene.2020.00586. PMID: 32733531; PMCID: PMC7358572.
47. Tan D J, Mitra M, Chiu A M, Coller H A. Intron retention is a robust marker of intertumoral heterogeneity in pancreatic ductal adenocarcinoma. NPJ Genom Med. 2020 Dec. 11;5(1):55. doi: 10.1038/s41525-020-00159-4. PMID: 33311498; PMCID: PMC7733475.
48. Inoue D, Polaski J T. Taylor J, Castel P, Chen S, Kobayashi S, Hogg S J, Hayashi Y, Pineda J M B, El Marabti E, Erickson C, Knorr K, Fukumoto M, Yamazaki H, Tanaka A, Fukui C, Lu S X, Durham B H, Liu B, Wang E, Mehta S, Zakheim D, Garippa R, Penson A, Chew G L, McCormick F, Bradley R K, Abdel-Wahab O. Minor intron retention drives clonal hematopoietic disorders and diverse cancer predisposition. Nat Genet. 2021 May;53(5):707-718. doi: 10.1038/s41588-021-00828-9. Epub 2021 Apr. 12. PMID: 33846634; PMCID: PMC8177065.
49. Dong C, Cesarano A, Bombaci G, Reiter J L, Yu C Y, Wang Y, Jiang Z, Zaid M A, Huang K, Lu X, Walker B A, Perna F, Liu Y. Intron retention-induced neoantigen load correlates with unfavorable prognosis in multiple myeloma. Oncogene. 2021 Oct;40(42):6130-6138. doi: 10.1038/s41388-021-02005-y. Epub 2021 Sep. 9. PMID: 34504297: PMCID: PMC8426332.
50. Zhang D, Hu Q, Liu X, Ji Y, Chao H P, Liu Y, Tracz A, Kirk J, Buonamici S, Zhu P, Wang J, Liu S, Tang D G. Intron retention is a hallmark and spliceosome represents a therapeutic vulnerability in aggressive prostate cancer. Nat Commun. 2020 Apr. 29;11(1):2089. doi: 10.1038/s41467-020-15815-7. PMID: 32350277; PMCID: PMC7190674.
51. Gil J, Esteban M. Induction of apoptosis by the dsRNA-dependent protein kinase (PKR): mechanism of action. Apoptosis. 2000 Apr;5(2):107-14. doi: 10.1023/a:1009664109241. PMID: 11232238.
52. Iordanov M S, Ryabinina O P, Schneider P. Magun B E. Two mechanisms of caspase 9 processing in double-stranded RNA- and virus-triggered apoptosis. Apoptosis. 2005 Jan;10(1):153-66. doi: 10.1007/s10495-005-6070-y. PMID: 15711931.
53. Takahashi K, Kawai T, Kumar H, Sato S, Yonehara S, Akira S. Roles of caspase-8 and caspase-10 in innate immune responses to double-stranded RNA. J Immunol. 2006 Apr. 15;176(8):4520-4. doi: 10.4049/jimmunol.176.8.4520. PMID: 16585540.
54. Sears N, Sen G C, Stark G R, Chattopadhyay S. Caspase-8-mediated cleavage inhibits IRF-3 protein by facilitating its proteasome-mediated degradation. J Biol Chem. 2011 Sep. 23;286(38):33037-44. doi: 10.1074/jbc.M111.257022. Epub 2011 Aug. 4. PMID: 21816816; PMCID: PMC3190878.
55. El Maadidi S, Faletti L, Berg B, Wenzl C, Wieland K, Chen Z J, Maurer U, Borner C. A novel mitochondrial MAVS/Caspase-8 platform links RNA virus-induced innate antiviral signaling to Bax/Bak-independent apoptosis. J Immunol. 2014 Feb. 1;192(3):1171-83. doi: 10.4049/jimmunol.1300842. Epub 2014 Jan. 3. PMID: 24391214.
56. Chen R, Ishak C A, De Carvalho D D. Endogenous Retroelements and the Viral Mimicry Response in Cancer Therapy and Cellular Homeostasis. Cancer Discov. 2021 Nov;11(11):2707-2725. doi: 10.1158/2159-8290.CD-21-0506. Epub 2021 Oct. 14. PMID: 34649957.
57. Chen-Cold and heterogenous T cell repertoire is associated with copy number aberrations and loss of immune genes in small-cell lung cancer-nature communication 2021.
58. Chiappinelli K B, Strissel P L, Desrichard A, Li H, Henke C, Akman B, Hein A, Rote N S, Cope L M, Snyder A, Makarov V, Budhu S, Slamon D J, Wolchok J D, Pardoll D M, Beckmann M W, Zahnow C A, Merghoub T, Chan T A, Baylin S B, Strick R. Inhibiting DNA Methylation Causes an Interferon Response in Cancer via dsRNA Including Endogenous Retroviruses. Cell. 2015 Aug. 27;162(5):974-86. doi: 10.1016/j.cell.2015.07.011. Erratum in: Cell. 2016 Feb. 25;164(5):1073. Buhu, Sadna [corrected to Budhu, Sadna]; Mergoub, Taha [corrected to Merghoub, Taha]. Erratum in: Cell. 2017 Apr. 6;169(2):361. PMID: 26317466; PMCID: PMC4556003.
59. Roulois D, Loo Yau H, Singhania R, Wang Y, Danesh A, Shen S Y, Han H, Liang G, Jones P A, Pugh T J, O'Brien C, De Carvalho D D. DNA-Demethylating Agents Target Colorectal Cancer Cells by Inducing Viral Mimicry by Endogenous Transcripts. Cell. 2015 Aug. 27;162(5):961-73. doi: 10.1016/j.cell.2015.07.056. PMID: 26317465; PMCID: PMC4843502.
60. Cañadas I, Thummalapalli R, Kim J W, Kitajima S, Jenkins R W, Christensen C L, Campisi M, Kuang Y, Zhang Y, Gjini E, Zhang G, Tian T, Sen D R, Miao D, Imamura Y, Thai T, Piel B, Terai H, Aref A R, Hagan T, Koyama S, Watanabe M, Baba H, Adeni A E, Lydon C A, Tamayo P, Wei Z, Herlyn M, Barbie T U, Uppaluri R, Sholl L M, Sicinska E, Sands J, Rodig S, Wong K K, Paweletz C P, Watanabe H, Barbie D A. Tumor innate immunity primed by specific interferon-stimulated endogenous retroviruses. Nat Med. 2018 Aug;24(8):1143-1150. doi: 10.1038/s41591-018-0116-5. Epub 2018 Jul. 23. PMID: 30038220; PMCID: PMC6082722.
61. Deblois G, Tonekaboni S A M, Grillo G, Martinez C, Kao Y I, Tai F, Ettayebi I, Fortier A M, Savage P, Fedor A N, Liu X, Guilhamon P, Lima-Fernandes E, Murison A, Kuasne H, Ba-Alawi W, Cescon D W, Arrowsmith C H, De Carvalho D D, Haibe-Kains B, Locasale J W, Park M, Lupien M. Epigenetic Switch-Induced Viral Mimicry Evasion in Chemotherapy-Resistant Breast Cancer. Cancer Discov. 2020 Sep;10(9):1312-1329. doi: 10.1158/2159-8290.CD-19-1493. Epub 2020 Jun. 16. PMID: 32546577.
62. Greenberg M V C, Bourc'his D. The diverse roles of DNA methylation in mammalian development and disease. Nat Rev Mol Cell Biol. 2019 Oct;20(10):590-607. doi: 10.1038/s41580-019-0159-6. Epub 2019 Aug. 9. PMID: 31399642.
63. Liu S, Brind'Amour J, Karimi M M, Shirane K, Bogutz A, Lefebvre L, Sasaki H, Shinkai Y, Lorincz M C. Setdb1 is required for germline development and silencing of H3K9me3-marked endogenous retroviruses in primordial germ cells. Genes Dev. 2014 Sep. 15;28(18):2041-55. doi: 10.1101/gad.244848.114. Erratum in: Genes Dev. 2015 Jan. 1;29(1):108. PMID: 25228647; PMCID: PMC4173156.
64. Shen J Z, Qiu Z, Wu Q, Finlay D, Garcia G, Sun D, Rantala J, Barshop W, Hope J L, Gimple R C, Sangfelt O, Bradley L M, Wohlschlegel J, Rich J N, Spruck C. FBXO44 promotes DNA replication-coupled repetitive element silencing in cancer cells. Cell. 2021 Jan. 21;184(2):352-369.e23. doi: 10.1016/j.cell.2020.11.042. Epub 2020 Dec. 23. PMID: 33357448; PMCID: PMC8043252.
65. Sheng W, LaFleur M W, Nguyen T H, Chen S, Chakravarthy A, Conway J R, Li Y, Chen H, Yang H, Hsu P H, Van Allen E M, Freeman G J, De Carvalho D D, He H H, Sharpe A H, Shi Y. LSD1 Ablation Stimulates Anti-tumor Immunity and Enables Checkpoint Blockade. Cell. 2018 Jul. 26;174(3):549-563.e19. doi: 10.1016/j.cell.2018.05.052. Epub 2018 Jun. 21. PMID: 29937226; PMCID: PMC6063761.
66. Leadem B R, Kagiampakis I, Wilson C, Cheung T K, Arnott D, Trojer P, Classon M, Easwaran H, Baylin S B. A KDM5 Inhibitor Increases Global H3K4 Trimethylation Occupancy and Enhances the Biological Efficacy of 5-Aza-2′-Deoxycytidine. Cancer Res. 2018 Mar. 1;78(5):1127-1139. doi: 10.1158/0008-5472.CAN-17-1453. Epub 2017 Dec. 27. PMID: 29282222; PMCID: PMC5935520.
67. Zhang S M, Cai W L, Liu X, Thakral D, Luo J, Chan L H, McGeary M K, Song E, Blenman K R M, Micevic G. Jessel S. Zhang Y, Yin M, Booth C J, Jilaveanu L B, Damsky W, Sznol M, Kluger H M, Iwasaki A, Bosenberg M W, Yan Q. KDM5B promotes immune evasion by recruiting SETDB1 to silence retroelements. Nature. 2021 Oct;598(7882):682-687. doi: 10.1038/s41586-021-03994-2. Epub 2021 Oct. 20. PMID: 34671158; PMCID: PMC8555464.
68. Stone M L, Chiappinelli K B, Li H, Murphy L M, Travers M E, Topper M J, Mathios D, Lim M, Shih I M, Wang T L, Hung C F, Bhargava V, Wiehagen K R, Cowley G S, Bachman K E, Strick R, Strissel P L, Baylin S B, Zahnow C A. Epigenetic therapy activates type I interferon signaling in murine ovarian cancer to reduce immunosuppression and tumor burden. Proc Natl Acad Sci U S A. 2017 Dec. 19;114(51):E10981-E10990. doi: 10.1073/pnas.1712514114. Epub 2017 Dec. 4. PMID: 29203668; PMCID: PMC5754782.
69. Ishak C A, De Carvalho D D. Reactivation of Endogenous Retroelements in Cancer Development and Therapy. Annu Rev Cancer Biol. 2020 Mar. 13;4:159-176. Doi: 10.1146/annurev-cancerbio-030419-033525.
70. Boukhaled G M, Harding S, Brooks D G. Opposing Roles of Type I Interferons in Cancer Immunity. Annu Rev Pathol. 2021 Jan. 24;16:167-198. doi: 10.1146/annurev-pathol-031920-093932. Epub 2020 Dec. 2. PMID: 33264572; PMCID: PMC8063563.
71. Espinet E, Gu Z, Imbusch C D, Giese N A, Büscher M, Safavi M, Weisenburger S. Klein C, Vogel V, Falcone M, Insua-Rodríguez J, Reitberger M, Thiel V, Kossi S O, Muckenhuber A, Sarai K, Lee A Y L, Backx E, Zarei S, Gaida M M, Rodríguez-Paredes M, Donato E, Yen H Y, Eils R, Schlesner M, Pfarr N, Hackert T, Plass C, Brors B, Steiger K, Weichenhan D. Arda H E, Rooman I, Kopp J L, Strobel O, Weichert W, Sprick M R, Trumpp A. Aggressive PDACs Show Hypomethylation of Repetitive Elements and the Execution of an Intrinsic IFN Program Linked to a Ductal Cell of Origin. Cancer Discov. 2021 Mar;11(3):638-659. doi: 10.1158/2159-8290.CD-20-1202. Epub 2020 Oct. 15. PMID: 33060108.
72. Maelfait J, Liverpool L, Bridgeman A, Ragan K B, Upton J W, Rehwinkel J. Sensing of viral and endogenous RNA by ZBP1/DAI induces necroptosis. EMBO J. 2017 Sep. 1;36(17):2529-2543. doi: 10.15252/embj.201796476. Epub 2017 Jul. 17. PMID: 28716805; PMCID: PMC5579359.
73. Kuriakose T, Kanneganti T D. ZBP1: Innate Sensor Regulating Cell Death and Inflammation. Trends Immunol. 2018 Feb;39(2):123-134. doi: 10.1016/j.it.2017.11.002. Epub 2017 Nov. 25. PMID: 29236673; PMCID: PMC5863909.
74. Balachandran S, Mocarski E S. Viral Z-RNA triggers ZBP1-dependent cell death. Curr Opin Virol. 2021 Dec;51:134-140. doi: 10.1016/j.coviro.2021.10.004. Epub 2021 Oct. 21. PMID: 34688984; PMCID: PMC8643332.
75. Chan F K, Luz N F. Moriwaki K. Programmed necrosis in the cross talk of cell death and inflammation. Annu Rev Immunol. 2015;33:79-106. doi: 10.1146/annurev-immunol-032414-112248. Epub 2014 Dec. 10. PMID: 25493335; PMCID: PMC4394030.
76. Lek M, Karczewski K J, Minikel E V, Samocha K E. Banks E, Fennell T, O'Donnell-Luria A H, Ware J S, Hill A J, Cummings B B, Tukiainen T, Birnbaum D P, Kosmicki J A, Duncan L E, Estrada K, Zhao F, Zou J, Pierce-Hoffman E, Berghout J, Cooper D N, Deflaux N, DePristo M, Do R, Flannick J, Fromer M, Gauthier L, Goldstein J, Gupta N, Howrigan D, Kiezun A, Kurki M I, Moonshine A L, Natarajan P, Orozco L, Peloso G M, Poplin R, Rivas M A, Ruano-Rubio V, Rose S A, Ruderfer D M, Shakir K, Stenson P D, Stevens C, Thomas B P, Tiao G, Tusie-Luna M T, Weisburd B, Won H H, Yu D, Altshuler D M, Ardissino D, Boehnke M, Danesh J, Donnelly S, Elosua R, Florez J C, Gabriel S B, Getz G, Glatt S J, Hultman C M, Kathiresan S, Laakso M, McCarroll S, McCarthy M I, McGovern D, McPherson R. Neale B M, Palotie A, Purcell S M, Saleheen D, Scharf J M, Sklar P, Sullivan P F, Tuomilehto J, Tsuang M T, Watkins H C, Wilson J G, Daly M J, MacArthur D G; Exome Aggregation Consortium. Analysis of protein-coding genetic variation in 60,706 humans. Nature. 2016 Aug. 18;536(7616):285-91. doi: 10.1038/nature19057. PMID: 27535533; PMCID: PMC5018207.
77. Zhou X, Edmonson M N, Wilkinson M R, Patel A, Wu G, Liu Y, Li Y, Zhang Z, Rusch M C, Parker M, Becksfort J, Downing J R, Zhang J. Exploring genomic alteration in pediatric cancer using ProteinPaint. Nat Genet. 2016 Jan;48(1):4-6. doi: 10.1038/ng.3466. PMID: 26711108; PMCID: PMC4892362.
78. Thapa R J, Ingram J P, Ragan K B, Nogusa S, Boyd D F, Benitez A A, Sridharan H, Kosoff R, Shubina M, Landsteiner V J, Andrake M, Vogel P, Sigal L J, tenOever B R, Thomas P G, Upton J W, Balachandran S. DAI Senses Influenza A Virus Genomic RNA and Activates RIPK3-Dependent Cell Death. Cell Host Microbe. 2016 Nov. 9;20(5):674-681. doi: 10.1016/j.chom.2016.09.014. Epub 2016 Oct. 13. PMID: 27746097; PMCID: PMC5687825.
79. Zhang T, Yin C, Boyd D F, Quarato G, Ingram J P, Shubina M, Ragan K B, Ishizuka T, Crawford J C, Tummers B, Rodriguez D A, Xue J, Peri S, Kaiser W J, López C B, Xu Y, Upton J W, Thomas P G, Green D R, Balachandran S. Influenza Virus Z-RNAs Induce ZBP1-Mediated Necroptosis. Cell. 2020 Mar. 19;180(6):1115-1129.e13. doi: 10.1016/j.cell.2020.02.050. PMID: 32200799; PMCID: PMC7153753.
80. Zhang T, Yin C, Fedorov A, Qiao L, Bao H. Beknazarov N. Wang S, Gautam A, Williams R M, Crawford J C, Peri S, Studitsky V, Beg A A, Thomas P G, Walkley C, Xu Y, Poptsova M, Herbert A, Balachandran S. ADAR1 masks the cancer immunotherapeutic promise of ZBP1-driven necroptosis. Nature. 2022 Jun;606(7914):594-602. doi: 10.1038/s41586-022-04753-7. Epub 2022 May 25. PMID: 35614224; PMCID: PMC9373927.
81. Jiao H, Wachsmuth L, Kumari S, Schwarzer R, Lin J, Eren R O, Fisher A, Lane R, Young G R, Kassiotis G, Kaiser W J, Pasparakis M. Z-nucleic-acid sensing triggers ZBP1-dependent necroptosis and inflammation. Nature. 2020 Apr;580(7803):391-395. doi: 10.1038/s41586-020-2129-8. Epub 2020 Mar. 25. Erratum in: Nature. 2020 Apr;580(7804):E10. PMID: 32296175; PMCID: PMC7279955.
82. Jiao H, Wachsmuth L, Wolf S, Lohmann J, Nagata M, Kaya G G, Oikonomou N, Kondylis V, Rogg M, Diebold M, Tröder S E, Zevnik B, Prinz M, Schell C, Young G R, Kassiotis G, Pasparakis M. ADAR1 averts fatal type I interferon induction by ZBP1. Nature. 2022 Jul;607(7920):776-783. doi: 10.1038/s41586-022-04878-9. Epub 2022 Jul. 20. PMID: 35859176; PMCID: PMC9329096.
83. Hubbard N W. Ames J M, Maurano M. Chu L H, Somfleth K Y, Gokhale N S, Werner M, Snyder J M. Lichauco K, Savan R, Stetson D B, Oberst A. ADAR1 mutation causes ZBP1-dependent immunopathology. Nature. 2022 Jul;607(7920):769-775. doi: 10.1038/s41586-022-04896-7. Epub 2022 Jul. 20. PMID: 35859177; PMCID: PMC9339495.
84. de Reuver R, Verdonck S, Dierick E, Nemegeer J, Hessmann E, Ahmad S, Jans M, Blancke G, Van Nieuwerburgh F, Botzki A, Vereecke L, van Loo G, Declercq W, Hur S. Vandenabeele P. Maelfait J. ADAR1 prevents autoinflammation by suppressing spontaneous ZBP1 activation. Nature. 2022 Jul;607(7920):784-789. doi: 10.1038/s41586-022-04974-w. Epub 2022 Jul. 20. PMID: 35859175.
85. Gong Y, Fan Z, Luo G, Yang C, Huang Q, Fan K, Cheng H, Jin K, Ni Q. Yu X. Liu C. The role of necroptosis in cancer biology and therapy. Mol Cancer. 2019 May 23;18(1):100. doi: 10.1186/s12943-019-1029-8. PMID: 31122251; PMCID: PMC6532150.
86. Choi M E. Price D R, Ryter S W, Choi A M K. Necroptosis: a crucial pathogenic mediator of human disease. JCI Insight. 2019 Aug. 8;4(15):e128834. doi: 10.1172/jci.insight.128834. PMID: 31391333; PMCID: PMC6693822.
87. Cretu C, Agrawal A A, Cook A. Will C L, Fekkes P, Smith P G, Lührmann R, Larsen N, Buonamici S, Pena V. Structural Basis of Splicing Modulation by Antitumor Macrolide Compounds. Mol Cell. 2018 Apr. 19;70(2):265-273.e8. doi: 10.1016/j.molcel.2018.03.011. Epub 2018 Apr. 12. PMID: 29656923.
88. Kotake Y, Sagane K, Owa T, Mimori-Kiyosue Y, Shimizu H, Uesugi M, Ishihama Y, Iwata M, Mizui Y. Splicing factor SF3b as a target of the antitumor natural product pladienolide. Nat Chem Biol. 2007 Sep;3(9):570-5. doi: 10.1038/nchembio.2007.16. Epub 2007 Jul. 22. PMID: 17643112.
89. Yokoi A, Kotake Y, Takahashi K, Kadowaki T, Matsumoto Y, Minoshima Y, Sugi N H, Sagane K, Hamaguchi M, Iwata M, Mizui Y. Biological validation that SF3b is a target of the antitumor macrolide pladienolide. FEBS J. 2011 Dec;278(24):4870-80. doi: 10.1111/j.1742-4658.2011.08387.x. Epub 2011 Oct. 31. PMID: 21981285.
90. Fujii K, Miyata Y, Takahashi 1, Koizumi H, Saji H, Hoshikawa M, Takagi M, Nishimura T, Nakamura H. Differential Proteomic Analysis between Small Cell Lung Carcinoma (SCLC) and Pulmonary Carcinoid Tumors Reveals Molecular Signatures for Malignancy in Lung Cancer. Proteomics Clin Appl. 2018 Nov;12(6):e1800015. doi: 10.1002/prca.201800015. Epub 2018 Jul. 5. PMID: 29888431.
91. Kraus A J, Brink B G, Siegel T N. Efficient and specific oligo-based depletion of rRNA. Sci Rep. 2019 Aug. 22;9(1):12281. doi: 10.1038/s41598-019-48692-2. PMID: 31439880; PMCID: PMC6706579.
92. Wilson D N. Doudna Cate J H. The structure and function of the eukaryotic ribosome. Cold Spring Harb Perspect Biol. 2012 May 1;4(5):a011536. doi: 10.1101/cshperspect.a011536. PMID: 22550233; PMCID: PMC3331703.
93. Grasso C S, Tsoi J. Onyshchenko M, Abril-Rodriguez G, Ross-Macdonald P, Wind-Rotolo M, Champhekar A, Medina E, Torrejon D Y, Shin D S, Tran P, Kim Y J, Puig-Saus C, Campbell K, Vega-Crespo A, Quist M, Martignier C, Luke J J, Wolchok J D, Johnson D B, Chmielowski B, Hodi F S, Bhatia S, Sharfman W, Urba W J, Slingluff C L Jr, Diab A, Haanen J B A G, Algarra S M, Pardoll D M, Anagnostou V, Topalian S L, Velculescu V E, Speiser D E, Kalbasi A, Ribas A. Conserved Interferon-y Signaling Drives Clinical Response to Immune Checkpoint Blockade Therapy in Melanoma. Cancer Cell. 2021 Jan. 11;39(1):122. doi: 10.1016/j.ccell.2020.11.015. Epub 2020 Dec. 10. Erratum for: Cancer Cell. 2020 Oct. 12;38(4):500-515.e3. PMID: 33306984; PMCID: PMC7885306.
94. Cui C, Xu C, Yang W. Chi Z, Sheng X, Si L, Xie Y, Yu J, Wang S, Yu R, Guo J, Kong Y. Ratio of the interferon-γ signature to the immunosuppression signature predicts anti-PD-1 therapy response in melanoma. NPJ Genom Med. 2021 Feb. 4;6(1):7. doi: 10.1038/s41525-021-00169-w. PMID: 33542239; PMCID: PMC7862369.
95. Corrales L, Matson V, Flood B. Spranger S, Gajewski T F. Innate immune signaling and regulation in cancer immunotherapy. Cell Res. 2017 Jan;27(1):96-108. doi: 10.1038/cr.2016.149. Epub 2016 Dec. 16. PMID: 27981969; PMCID: PMC5223230.
96. Schumacher T N, Schreiber R D. Neoantigens in cancer immunotherapy. Science. 2015 Apr. 3;348(6230):69-74. doi: 10.1126/science.aaa4971. PMID: 25838375.
97. Sharma P, Hu-Lieskovan S, Wargo J A, Ribas A. Primary, Adaptive, and Acquired Resistance to Cancer Immunotherapy. Cell. 2017 Feb. 9;168(4):707-723. doi: 10.1016/j.cell.2017.01.017. PMID: 28187290; PMCID: PMC5391692.
98. Rodriguez de la Fuente L, Law A M K, Gallego-Ortega D, Valdes-Mora F. Tumor dissociation of highly viable cell suspensions for single-cell omic analyses in mouse models of breast cancer. STAR Protoc. 2021 Sep. 17;2(4):100841. doi: 10.1016/j.xpro.2021.100841. PMID: 34585168; PMCID: PMC8456108.
99. Fouad Y A, Aanei C. Revisiting the hallmarks of cancer. Am J Cancer Res. 2017 May 1;7(5):1016-1036. PMID: 28560055; PMCID: PMC5446472.
100. Lu S X, De Neef E, Thomas J D, Sabio E, Rousseau B, Gigoux M, Knorr D A, Greenbaum B, Elhanati Y, Hogg S J, Chow A, Ghosh A, Xie A, Zamarin D, Cui D, Erickson C, Singer M, Cho H, Wang E, Lu B, Durham B H, Shah H, Chowell D, Gabel A M, Shen Y, Liu J, Jin J, Rhodes M C, Taylor R E, Molina H, Wolchok J D, Merghoub T, Diaz L A Jr, Abdel-Wahab O, Bradley R K. Pharmacologic modulation of RNA splicing enhances anti-tumor immunity. Cell. 2021 Jul. 22;184(15):4032-4047.e31. doi: 10.1016/j.cell.2021.05.038. Epub 2021 Jun. 24. PMID: 34171309: PMCID: PMC8684350.
101. Jenkins R W, Aref A R, Lizotte P H, Ivanova E, Stinson S, Zhou C W, Bowden M, Deng J, Liu H, Miao D, He M X, Walker W, Zhang G, Tian T, Cheng C, Wei Z, Palakurthi S, Bittinger M, Vitzthum H, Kim J W, Merlino A, Quinn M, Venkataramani C, Kaplan J A, Portell A, Gokhale P C, Phillips B, Smart A, Rotem A, Jones R E, Keogh L, Anguiano M, Stapleton L, Jia Z, Barzily-Rokni M, Cañadas I, Thai T C, Hammond M R, Vlahos R, Wang E S, Zhang H, Li S, Hanna G J, Huang W, Hoang M P, Piris A, Eliane J P, Stemmer-Rachamimov A O, Cameron L, Su M J, Shah P, Izar B, Thakuria M, LeBoeuf N R, Rabinowits G. Gunda V, Parangi S, Cleary J M, Miller B C, Kitajima S, Thummalapalli R, Miao B, Barbie T U, Sivathanu V, Wong J, Richards W G, Bueno R, Yoon C H, Miret J, Herlyn M, Garraway L A, Van Allen E M, Freeman G J, Kirschmeier P T, Lorch J H, Ott P A, Hodi F S, Flaherty K T, Kamm R D, Boland G M, Wong K K, Dornan D, Paweletz C P, Barbie D A. Ex Vivo Profiling of PD-1 Blockade Using Organotypic Tumor Spheroids. Cancer Discov. 2018 Feb;8(2):196-215. doi: 10.1158/2159-8290.CD-17-0833. Epub 2017 Nov. 3. PMID: 29101162; PMCID: PMC5809290.
102. Aref A R, Campisi M, Ivanova E, Portell A, Larios D, Piel B P, Mathur N, Zhou C, Coakley R V, Bartels A, Bowden M, Herbert Z, Hill S, Gilhooley S, Carter J, Cañadas I, Thai T C, Kitajima S, Chiono V, Paweletz C P, Barbie D A, Kamm R D, Jenkins R W. 3D microfluidic ex vivo culture of organotypic tumor spheroids to model immune checkpoint blockade. Lab Chip. 2018 Oct. 9;18(20):3129-3143. doi: 10.1039/c81c00322j. PMID: 30183789; PMCID: PMC6274590.
103. Kitajima S. Ivanova E, Guo S, Yoshida R, Campisi M, Sundararaman S K, Tange S, Mitsuishi Y, Thai T C, Masuda S, Piel B P, Sholl L M, Kirschmeier P T, Paweletz C P, Watanabe H, Yajima M, Barbie D A. Suppression of STING Associated with LKB1 Loss in KRAS-Driven Lung Cancer. Cancer Discov. 2019 Jan;9(1):34-45. doi: 10.1158/2159-8290.CD-18-0689. Epub 2018 Oct. 8. PMID: 30297358; PMCID: PMC6328329.
104. Schwartz T, Behlke J, Lowenhaupt K, Heinemann U, Rich A. Structure of the DLM-1-Z-DNA complex reveals a conserved family of Z-DNA-binding proteins. Nat Struct Biol. 2001 Sep;8(9):761-5. doi: 10.1038/nsb0901-761. PMID: 11524677.
105. Ha S C, Van Quyen D, Hwang H Y, Oh D B, Brown ^{B} A 2nd, Lee S M, Park H J, Ahn J H, Kim K K, Kim Y G. Biochemical characterization and preliminary X-ray crystallographic study of the domains of human ZBP1 bound to left-handed Z-DNA. Biochim Biophys Acta. 2006 Feb;1764(2):320-3. doi: 10.1016/j.bbapap.2005.12.012. Epub 2006 Jan. 18. PMID: 16448869.
106. Bae S, Kim Y, Kim D, Kim K K, Kim Y G, Hohng S. Energetics of Z-DNA binding protein-mediated helicity reversals in DNA, RNA, and DNA-RNA duplexes. J Phys Chem B. 2013 Nov. 7;117(44):13866-71. doi: 10.1021/jp409862j. Epub 2013 Oct. 28. PMID: 24111542.
107. Herbert A, Alfken J, Kim Y G, Mian I S, Nishikura K, Rich A. A Z-DNA binding domain present in the human editing enzyme, double-stranded RNA adenosine deaminase. Proc Natl Acad Sci U S A. 1997 Aug. 5;94(16):8421-6. doi: 10.1073/pnas.94.16.8421. PMID: 9237992; PMCID: PMC22942.
108. Brown ^{B} A 2nd, Lowenhaupt K, Wilbert C M, Hanlon E B, Rich A. The zalpha domain of the editing enzyme dsRNA adenosine deaminase binds left-handed Z-RNA as well as Z-DNA. Proc Natl Acad Sci U S A. 2000 Dec. 5;97(25):13532-6. doi: 10.1073/pnas.240464097. PMID: 11087828; PMCID: PMC17610.
109. Oh D B, Kim Y G, Rich A. Z-DNA-binding proteins can act as potent effectors of gene expression in vivo. Proc Natl Acad Sci U S A. 2002 Dec. 24;99(26):16666-71. doi: 10.1073/pnas.262672699. Epub 2002 Dec. 16. PMID: 12486233; PMCID: PMC139201.
110. Chan F K, Luz N F, Moriwaki K. Programmed necrosis in the cross talk of cell death and inflammation. Annu Rev Immunol. 2015;33:79-106. doi: 10.1146/annurev-immunol-032414-112248. Epub 2014 Dec. 10. PMID: 25493335; PMCID: PMC4394030.
111. Lafont E, Draber P, Rieser E, Reichert M, Kupka S, de Miguel D, Draberova H, von Mässenhausen A, Bhamra A, Henderson S, Wojdyla K, Chalk A, Surinova S, Linkermann A, Walczak H. TBK1 and IKKE prevent TNF-induced cell death by RIPK1 phosphorylation. Nat Cell Biol. 2018 Dec;20(12):1389-1399. doi: 10.1038/s41556-018-0229-6. Epub 2018 Nov. 12. PMID: 30420664; PMCID: PMC6268100.
112. Taft J, Markson M, Legarda D, Patel R, Chan M, Malle L, Richardson A, Gruber C, Martin-Fernández M, Mancini G M S, van Laar J A M, van Pelt P, Buta S, Wokke B H A, Sabli I K D, Sancho-Shimizu V, Chavan P P, Schnappauf O, Khubchandani R, Cüceoğlu M K, Özen S, Kastner D L, Ting A T, Aksentijevich I, Hollink I H I M, Bogunovic D. Human TBK1 deficiency leads to autoinflammation driven by TNF-induced cell death. Cell. 2021 Aug. 19;184(17):4447-4463.e20. doi: 10.1016/j.cell.2021.07.026. Epub 2021 Aug. 6. PMID: 34363755: PMCID: PMC8380741.
113. Vredevoogd D W, Kuilman T, Ligtenberg M A, Boshuizen J, Stecker K E, de Bruijn B, Krijgsman O, Huang X, Kenski J C N, Lacroix R, Mezzadra R, Gomez-Eerland R, Yildiz M, Dagidir I, Apriamashvili G, Zandhuis N, van der Noort V, Visser N L, Blank C U, Altelaar M, Schumacher T N, Peeper D S. Augmenting Immunotherapy Impact by Lowering Tumor TNF Cytotoxicity Threshold. Cell. 2019 Jul. 25;178(3):585-599.e15. doi: 10.1016/j.cell.2019.06.014. Epub 2019 Jul. 11. Erratum in: Cell. 2020 Jan. 23;180(2):404-405. PMID: 31303383.
114. Xu D, Jin T, Zhu H, Chen H, Ofengeim D, Zou C, Mifflin L, Pan L, Amin P, Li W, Shan B, Naito M G, Meng H, Li Y, Pan H, Aron L, Adiconis X, Levin J Z, Yankner B A, Yuan J. TBK1 Suppresses RIPK1-Driven Apoptosis and Inflammation during Development and in Aging. Cell. 2018 Sep. 6;174(6):1477-1491.e19. doi: 10.1016/j.cell.2018.07.041. Epub 2018 Aug. 23. PMID: 30146158; PMCID: PMC6128749.

While certain features of the invention have been described herein, many modifications, substitutions, changes, and equivalents will now occur to those of ordinary skill in the art. It is, therefore, to be understood that the appended claims are intended to cover all such modifications and changes as fall within the scope of the invention.

1. A method for inducing ZBP1-dependent necroptosis in Small Cell Lung Cancer (SCLC) tumor cells, comprising administering to said subject effective amounts at least one spliceosome modulator and at least one immunotherapy, thereby reducing tumor cell proliferation or inducing tumor cell killing which exceeds that observed in tumor cells treated with the modulator alone,
wherein said spliceosome modulator is selected from pladienolide B, H3B-8800, sudemycin D6, and indisulam; and said immunotherapy is a PD-1 inhibitor or PD-L1 inhibitor.

2-3. (canceled)

3. The method of claim 1, wherein the spliceosome modulator is pladienolide B which is administered
a) via intratumoral administration at a dose of 0-200 nM;
b) via intraperitoneal administration at a dose of 0-20 mg/kg; or
c) via systemic or intravenous administration at a dose of 2-20 mg/kg.

4-5. (canceled)

6. The method of claim 1, wherein the splicosome modulator is H3B-8800 which is administered orally at a dose of 0-40 mg.

7. The method of claim 1, wherein the spliceosome modulator is sudemycin D6 which is administered via systemic or intravenous administration at a dose of 14 mg/kg-50 mg/kg.

8-9. (canceled)

10. The method of claim 1, wherein the spliceosome modulator is indisulam which is administered via systemic or intravenous administration at a dose of 12.5 mg/kg-100 mg/kg.

11. The method of claim 1, wherein said SCLC is a COLD cancer and said modulators are administered in a pharmaceutically acceptable carrier via route selected from systemic, oral, intraperitoneal, intravenous, intracerebral, intratumoral and topical administration, said method optionally comprising administration of a radiotherapy.

12. A method for treating a subject having tumors comprising, administering to said subject at least one spliceosome modulator, thereby reducing tumor cell proliferation or inducing tumor cell killing which exceeds that observed in tumor cells not treated with the modulator.

13. The method of claim 12, wherein said at least one spliceosome modulator is selected from a SF3b inhibitor, an SF3B1 inhibitor, or a TIME modulator.

14. The method of claim 12, wherein said spliceosome modulator is selected from at least one of pladienolide D, pladienolide B, pladienolide A, pladienolide C, pladienolide E, pladienolide F, pladienolide G, H3B-8800, indisulam, herboxidiene, spliceostatin, sudemycin, FR901463, FR901464, FR901465, splicostatins A-G, thailanstatins A-C, meamycins, E7107, FD-895, herboxidiene, GEX1A, GEX1Q1-5, RON-18690A (18-deoxyherboxidiene), sudemycins C1, sudemycins D1, sudemycins F1, sudemycins E, sudemycins D6, isoginkgetin, madrasin, tetrocarcin A, N-palmitoyl-L-leucine, psoromic acid, clotrimazole, NSC635326, napthazarin and/or derivatives thereof.

15. (canceled)

16. The method of claim 12, further comprising administering at least one additional cancer treatment or therapeutic agent.

17. The method of claim 16, wherein the therapeutic agent or additional cancer treatment is a PD-L1 inhibitor, immunotherapy, TNF therapy, chemotherapy, and/or radiotherapy.

18-20. (canceled)

21. The method of claim 12, wherein the tumor is a recalcitrant tumor, a COLD cancer, a lung cancer, or a Small Cell Lung Cancer (SCLC).

22-28. (canceled)

29. A method of enhancing tumor vulnerability to chemotherapy or immunotherapy in a tumor with a low mutational burden, the method comprising administering at least one spliceosome modulator, thereby reducing tumor cell proliferation.

30. The method of claim 29, further comprising administering at least one chemotherapy or immunotherapy, thereby inducing tumor cell killing which exceeds that observed in tumor cells treated with the spliceosome modulator alone.

31-34. (canceled)

35. The method of claim 29, wherein said at least one spliceosome modulator is selected from a SF3b inhibitor, an SF3B1 inhibitor, or a TIME modulator.

36. The method of claim 35, wherein said spliceosome modulator is selected from pladienolide D, pladienolide B, pladienolide A, pladienolide C, pladienolide E, pladienolide F, pladienolide G, H3B-8800, indisulam, herboxidiene, spliceostatin, sudemycin, FR901463, FR901464, FR901465, splicostatins A-G, thailanstatins A-C, meamycins, E7107, FD-895, herboxidiene, GEX1A, GEX1Q1-5, RON-18690A (18-deoxyherboxidiene), sudemycins C1, sudemycins D1, sudemycins F1, sudemycins E, sudemycins D6, isoginkgetin, madrasin, tetrocarcin A, N-palmitoyl-L-leucine, psoromic acid, clotrimazole, NSC635326, napthazarin and/or derivatives thereof.

37-38. (canceled)

39. The method of claim 29, wherein said chemotherapy or immunotherapy is a PD-L1 inhibitor or a PD-1 inhibitor.

40. The method of claim 29, wherein the tumor is a recalcitrant tumor, a COLD cancer, a lung cancer, or a Small Cell Lung Cancer (SCLC).

41-44. (canceled)

45. The method of claim 1 wherein the cells are at least one of non-tumorous control cells and tumor cells.

46. (canceled)

47. The method of claim 1, further comprising administering at least one TNFR1, TRADD, FADD, or CASP8 inhibitor.

48-49. (canceled)
